SEQUENCE LISTING (32 sequences)

SEQ ID NO: 1 - RNA, 19 nt, Artificial Sequence
  Synthesized
ugacaaacgggcaacauac

SEQ ID NO: 2 - RNA, 19 nt, Artificial Sequence
  Synthesized
guauguugcccguuuguca

SEQ ID NO: 3 - RNA, 19 nt, Artificial Sequence
  Synthesized
guauguugcccguuuguca

SEQ ID NO: 4 - RNA, 19 nt, Artificial Sequence
  Synthesized
guauguugcccguuuguca

SEQ ID NO: 5 - RNA, 19 nt, Artificial Sequence
  Synthesized
guauguugcccguuuguca

SEQ ID NO: 6 - RNA, 19 nt, Artificial Sequence
  Synthesized
guauguugcccguuuguca

SEQ ID NO: 7 - RNA, 19 nt, Artificial Sequence
  Synthesized
guauguugcccguuuguca

SEQ ID NO: 8 - RNA, 19 nt, Artificial Sequence
  Synthesized
guauguugcccguuuguca

SEQ ID NO: 9 - RNA, 19 nt, Artificial Sequence
  Synthesized
guauguugcccguuuguca

SEQ ID NO: 10 - RNA, 19 nt, Artificial Sequence
  Synthesized
guauguugcccguuuguca

SEQ ID NO: 11 - RNA, 19 nt, Artificial Sequence
  Synthesized
guauguugcccguuuguca

SEQ ID NO: 12 - RNA, 19 nt, Artificial Sequence
  Synthesized
guauguugcccguuuguca

SEQ ID NO: 13 - RNA, 19 nt, Artificial Sequence
  Synthesized
guauguugcccguuuguca

SEQ ID NO: 14 - RNA, 19 nt, Artificial Sequence
  Synthesized
cauacaacgggcaaacagu

SEQ ID NO: 15 - RNA, 19 nt, Artificial Sequence
  Synthesized
guauguugcccguuuguca

SEQ ID NO: 16 - RNA, 19 nt, Artificial Sequence
  Synthesized
cauacaacgggcaaacagu

SEQ ID NO: 17 - RNA, 19 nt, Artificial Sequence
  Synthesized
guauguugcccguuuguca

SEQ ID NO: 18 - RNA, 19 nt, Artificial Sequence
  Synthesized
cauacaacgggcaaacagu

SEQ ID NO: 19 - RNA, 19 nt, Artificial Sequence
  Synthesized
guauguugcccguuuguca

SEQ ID NO: 20 - RNA, 19 nt, Artificial Sequence
  Synthesized
cauacaacgggcaaacagu

SEQ ID NO: 21 - RNA, 19 nt, Artificial Sequence
  Synthesized
guauguugcccguuuguca

SEQ ID NO: 22 - RNA, 19 nt, Artificial Sequence
  Synthesized
cauacaacgggcaaacagu

SEQ ID NO: 23 - RNA, 19 nt, Artificial Sequence
  Synthesized
guauguugcccguuuguca

SEQ ID NO: 24 - RNA, 19 nt, Artificial Sequence
  Synthesized
cauacaacgggcaaacagu

SEQ ID NO: 25 - RNA, 19 nt, Artificial Sequence
  Synthesized
guauguugcccguuuguca

SEQ ID NO: 26 - RNA, 19 nt, Artificial Sequence
  Synthesized
cauacaacgggcaaacagu

SEQ ID NO: 27 - RNA, 19 nt, Artificial Sequence
  Synthesized
guauguugcccguuuguca

SEQ ID NO: 28 - RNA, 19 nt, Artificial Sequence
  Synthesized
cauacaacgggcaaacagu

SEQ ID NO: 29 - RNA, 19 nt, Artificial Sequence
  Synthesized
guauguugcccguuuguca

SEQ ID NO: 30 - RNA, 19 nt, Artificial Sequence
  Synthesized
cauacaacgggcaaacagu

SEQ ID NO: 31 - RNA, 19 nt, Artificial Sequence
  Synthesized
guauguugcccguuuguca

SEQ ID NO: 32 - RNA, 19 nt, Artificial Sequence
  Synthesized
cauacaacgggcaaacagu

(19) United States
(12) Patent Application Publication
(10) Pub. No.: US 2023/0293701 A1
(43) Pub. Date: Sep. 21, 2023

(54) LIGAND COMPOUNDS, CONJUGATES, AND APPLICATIONS THEREOF

(71) Applicant: ARGORNA PHARMACEUTICALS LTD, Guangzhou, Guangdong (CN)

(72) Inventors: Bill Biliang ZHANG, Boston, MA (US); Haoting ZHAO, Guangzhou, Guangdong (CN)

(21) Appl. No.: 18/009,742

(22) PCT Filed: Jan. 20, 2021
(86) PCT No.: PCT/CN2021/072887

§ 371 Date: Dec. 12, 2022

(51) Int. Cl. A61K 47/54; C12N 15/113; A61P 1/16; C07H 15/26; C07H 15/18

(52) U.S. Cl. CPC A61K 47/549; C12N 15/113; A61P 1/16; C07H 15/26; C07H 15/18; C12N 2310/351; C12N 2310/11

(57) ABSTRACT

This disclosure features chemical entities (e.g., a compound or a pharmaceutically acceptable salt, and/or hydrate, and/or cocrystal, and/or drug combination of the compound) that comprise one or more ligand moieties for an asialoglycoprotein receptor (ASGPR). Exemplary chemical entities can further comprise an oligonucleotide. Said chemical entities are useful, e.g., in the targeted delivery of oligonucleotides to liver cells (e.g., liver parenchymal cells). The chemical entities are useful e.g., in the treatment of conditions or diseases caused by the expression (e.g., abnormal expression) of one or more genes in liver cells This disclosure also features compositions containing the same as well as methods of using and making the same.

### SEQUENCE LISTING

The present application contains a Sequence Listing which has been submitted electronically in ASCII format. Said ASCII copy is named_Sequence_Listing.txt and is 6,125 bytes in size, and contains 32 sequences from SEQ ID NO:1 to SEQ ID NO:32 described in examples of this file.

### TECHNICAL FIELD

This disclosure relates to the technological area of nucleic acid delivery. Ligand compounds, oligonucleotide conjugates, and methods of making and using the same are disclosed.

### BACKGROUND

Asialoglycoprotein receptor (ASGPR) is an abundant endocylic receptor of hetero-oligomers, which exists mainly on the surface of the cell membrane of liver parenchymal cells facing the side of sinusoidal space and has specificity for sugar. As the terminal sialic acid of the glycoproteins is removed through hydrolysis by enzymes or acidolysis, the exposed penultimates are galactose residues. Therefore, the sugar-binding specificity of ASGPR is actually galactosyl, and it is also called galactose-specific receptor. ASGPRs are mainly distributed in the liver parenchymal cells, and low in content in other cells. As such, the ASGPRs provide possible receptors for liver targeted transport.

Glycoproteins terminated with non-reducing galactose (Gal) or N-acetylgalactosamine (GalNAc) residues can be recognized by ASGPRs, wherein the affinity of GalNAc to ASGPR is about 50 times higher than that of Gal (Iobst S T et al, J Biol Chem. 1996, 271 (12) 6686-6693). In vitro experiments show that the affinity of clustered sugar residues is much higher than that of non-clustered sugar residues by simultaneously occupying the binding sites of the receptor with an affinity order of tetraantennary>triantennal>>biantennal>>monoantennal galactosides (Lee Y C, et al, J Biol Chem, 1983, 258 (1): 199-202).

ASGPR receptor-mediated liver targeting oligonucleotide is a new breakthrough in the research field of nucleic acid innovative drugs. In 2012, Alnylam Pharmaceuticals Inc. covalently linked triantennary GalNAc structure previously studied with small interfering RNA (siRNA) to achieve liver-targeted delivery of siRNA in vivo. Using this technology, researchers have developed drugs for amyloidosis, hemophilia, hypercholesterolemia, liver porphyrin, hepatitis B and other diseases. In 2019, the first GalNAc-siRNA drug was approved while two additional drugs are seeking approval. Over ten drug candidates have entered into clinical studies (http://www.alnylam.com/product-pipeline/). In 2014, ISIS Pharmaceuticals of the United States covalently linked triantennary GalNAc and antisense nucleic acid to achieve liver-targeted drug delivery in animals, with 10-fold increase in antisense nucleic acid activity after linking (Prakash, T. P. et al, Nucleic Acids Res. 42, 8796-807).

### SUMMARY

This disclosure features chemical entities (e.g., a compound or a pharmaceutically acceptable salt, and/or hydrate, and/or cocrystal, and/or drug combination of the compound) that comprise one or more ligand moieties for an asialoglycoprotein receptor (ASGPR). Exemplary chemical entities can further comprise an oligonucleotide. Said chemical entities are useful, e.g., in the targeted delivery of oligonucleotides to liver cells (e.g., liver parenchymal cells). The chemical entities are useful e.g., in the treatment of conditions or diseases caused by the expression (e.g., abnormal expression) of one or more genes in liver cells This disclosure also features compositions containing the same as well as methods of using and making the same.

In one aspect, this disclosure features compounds of Formula (I):

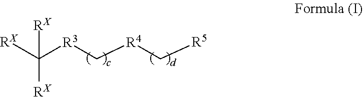

or a pharmaceutically acceptable salt thereof, wherein: R^{X}, R³, c, R⁴, d, and R⁵ can be as defined anywhere herein.

In some embodiments, the compound of Formula (I) is a conjugate compound of Formula (II):

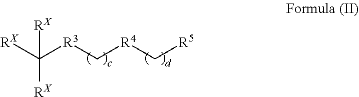

or a pharmaceutically acceptable salt thereof, wherein:

R⁵ is

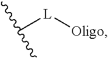

wherein Oligo is an oligonucleotide that is attached to L via the 5′-end, 3′-end, or sequence middle of any strand via a phosphate group; and

-L-, R^{X}, R³, c, R⁴, and d can be as defined for Formula (I) anywhere herein.

In another aspect, provided herein are pharmaceutical compositions comprising a chemical entity as described herein (e.g., a compound of Formula (II) or a pharmaceutically acceptable salt thereof) and a pharmaceutically acceptable excipient.

In another aspect, provided herein are methods for treating and/or preventing pathological conditions or diseases in a subject, wherein the conditions or diseases are caused by the expression of one or more genes in liver cells, the method comprising administering to the subject a chemical entity as described herein (e.g., a compound of Formula (II) or a pharmaceutically acceptable salt thereof); or a pharmaceutical composition comprising a chemical entity as described herein (e.g., a compound of Formula (II) or a pharmaceutically acceptable salt thereof), and a pharmaceutically acceptable excipient.

In a further aspect, provided herein are methods for detecting or localizing RNA in the liver of a subject, comprising administering to the subject a chemical entity as described herein (e.g., a compound of Formula (II) or a pharmaceutically acceptable salt thereof); or a pharmaceutical composition comprising a chemical entity as described herein (e.g., a compound of Formula (II) or a pharmaceutically acceptable salt thereof), and a pharmaceutically acceptable excipient.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “oligonucleotide” refers to an oligomeric compound containing a plurality of linked chemically modified or unmodified nucleotides having a length of less than about 100 nucleotides, such as, e.g., 1-20 nucleotides, 20-40 nucleotides, 40-60 nucleotides, 60-80 nucleotides, 80-100 nucleotides, or 1-50 nucleotides. In certain embodiments, the oligonucleotide can include a non-nucleic acid conjugate group. In certain embodiments, the oligonucleotide comprises ribonucleic acid (RNA), deoxyribonucleic acid (DNA), or peptide nucleic acid (PNA). In certain embodiments, the oligonucleotide is double-stranded or single-stranded. In certain embodiments, the oligonucleotide is an siRNA, an aptamer, an antisense nucleic acid, an sgRNA, a tractRNA, or crRNA.

As used herein, the term “conjugate” or “conjugate group” means an atom or atomic group bound to an oligonucleotide. In some cases, the conjugate groups alter one or more properties of the oligonucleotide to which they are linked, including but not limited to pharmacodynamics, pharmacokinetics, binding, absorption, cell distribution, cell uptake, charge, and/or clearance properties.

As used herein, the term “receptor” refers to a biological macromolecule composed of glycoproteins or lipoproteins, present in the cell membrane, cytoplasm, or nucleus of a cell, with different receptors having specific structures and configurations. As used herein, the term “ligand” refers to a substance that has the ability to recognize and bind to a receptor. In certain embodiments, the ligand is a ligand having affinity for an asialoglycoprotein receptor (ASGPR). In certain embodiments, the ligand is a carbohydrate, such as monosaccharides and polysaccharides, including but not limited to: galactose, N-acetylgalactosamine, mannose, glucose, glucosamine and fucose.

As used herein, the term “polysaccharide” refers to a polymer formed from a plurality of monosaccharide groups linked by glycosidic linkages. In the present disclosure, polysaccharides include oligoses and oligosaccharides. Generally, “oligose” refers to a polymer composed of 2-10 monosaccharide groups linked by glycosidic bonds, and “oligosaccharide” refers to a polymer composed of fewer than 20 monosaccharide groups linked by glycosidic bonds.

As used herein, the term “about” should be understood by those skilled in the art and will vary to some extent depending on the context in which it is used. If, depending on the context in which the term is used, its meaning is not clear to those skilled in the art, then the meaning of “about” is such that the deviation does not exceed plus or minus 10% of the specified value or range.

As used herein, the term “preventing” refers to preventing or delaying the onset of a disease.

As used herein, the term “treating” refers to curing or at least partially arresting the progression of a disease, or alleviating the symptoms of a disease.

As used herein, the term “effective amount” refers to an amount effective to achieve the intended purpose. For example, an amount effective to prevent a disease refers to an amount effective to prevent, arrest, or delay the onset of the disease. Determination of such effective amounts is within the ability of one skilled in the art.

The term “acceptable” with respect to a formulation, composition or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and omithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “gene-related disease” refers to a disease that results from an abnormal expression of one or more genes and/or an abnormal activity of proteins expressed by these genes. Similarly, these genes are known as disease-related genes.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo (e.g., —CF₃).

The term “alkoxy” refers to an —O-alkyl radical (e.g., —OCH₃).

The term “alkylene” refers to a divalent alkyl (e.g., —CH₂—).

The term “alkenyl” refers to a hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein includes non-aromatic cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Cycloalkyl groups may have any degree of saturation provided that none of the rings in the ring system are aromatic. Accordingly, cycloalkyl can be fully saturated. Non-limiting examples include: cyclopropyl, cyclobutyl, cyclopentyl, cyclohexyl, cycloheptyl, and cyclooctyl. Cycloalky can also include partially unsaturated cyclic hydrocarbon groups having 3 to 20 ring carbons, preferably 3 to 16 ring carbons, and more preferably 3 to 12 ring carbons or 3-10 ring carbons or 3-6 ring carbons. Non-limiting examples can include cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

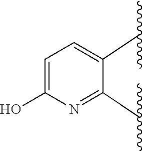 [SMILES: *c1ccc(O)nc1*]

encompasses the tautomeric form containing the moiety:

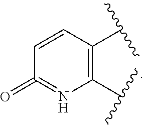

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or drug combination of the compound) that comprise one or more ligand moieties for an asialoglycoprotein receptor (ASGPR). Exemplary chemical entities can further comprise an oligonucleotide. Said chemical entities are useful, e.g., in the targeted delivery of oligonucleotides to liver cells (e.g., liver parenchymal cells). The chemical entities are useful e.g., in the treatment of conditions or diseases caused by the expression (e.g., abnormal expression) of one or more genes in liver cells This disclosure also features compositions containing the same as well as methods of using and making the same.

In one aspect, this disclosure provides compounds of Formula (I):

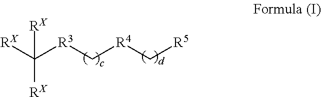

or a pharmaceutically acceptable salt thereof, wherein:

each R^{X} is independently selected from the group consisting of:
H;
—CH₂OR^{X2}, wherein R^{X2} is H, C₁₋₆ alkyl, or a hydroxyl protecting group; and

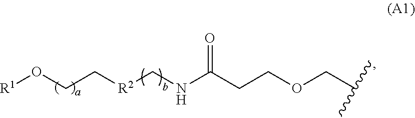

provided that at least one R^{X} is a group of Formula (A1);

each R¹ is an independently selected moiety capable of binding an asialoglycoproteinreceptor (ASGPR);

each R² is independently selected from the group consisting of: —C(R⁶)₂—; —OC(R⁶)₂C(R⁶)₂O—; —C(R⁶)(OH)—C(R⁶)(OH)—; *—C(═O)NR⁷—; *—NR⁷C(═O)—; C₆₋₁₀ arylene; C₂₋₆ alkenylene; and C₂₋₆ alkynylene,

wherein the C₆₋₁₀ arylene, C₂₋₆ alkenylene, and C₂₋₆ alkynylene are each optionally substituted with 1-4 independently selected R^{a}, and the * represents the point of attachment to

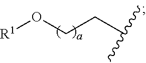

R³ is selected from the group consisting of:

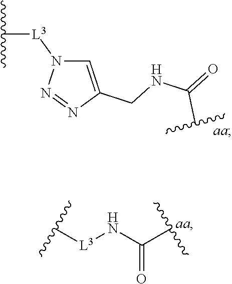

wherein aa represents the point of attachment to

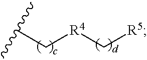

—(CR⁶R⁶)ₓ—O—(CR⁶R⁶)_{y}—, wherein x and y are independently 0, 1, 2, or 3; and
-L³-L^{3C}-, wherein L^{3C} is selected from the group consisting of: C₃₋₁₀ cycloalkylene, C₆₋₁₀ arylene, 5-10 membered heteroarylene, and 4-10 membered heterocyclylene, each of which is optionally substituted with 1-4 independently selected R^{a};

-L³ is —C(R⁶)₂—,

R⁴ is selected from the group consisting of: —C(R⁶)₂—; —OC(R⁶)₂C(R⁶)₂O—; *—C(═O)NR⁷—; *—NR⁷C(═O)—; C₆₋₁₀ arylene, C₃₋₁₀ cycloalkylene, 5-10 membered heteroarylene, and 4-10 membered heterocyclylene,

wherein the C₆₋₁₀ arylene, C₃₋₁₀ cycloalkylene, 5-10 membered heteroarylene, and 4-10 membered heterocyclylene are each optionally substituted with 1-4 independently selected R^{a}, and

wherein the * represents the point of attachment to

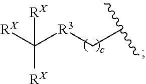

R⁵ is selected from the group consisting of:
hydroxyl; C(O)OH;

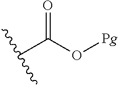

wherein Pg is a carboxyl activating group or a carboxyl protecting group;

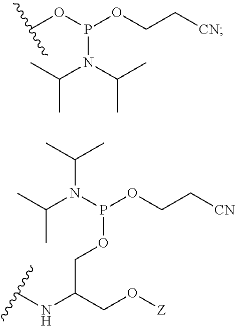

wherein Z is a hydroxyl protecting group,

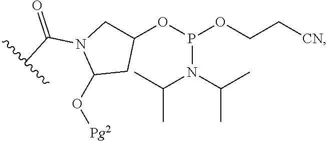

wherein Pg² is a hydroxyl protecting group; and

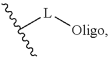

wherein: L is a bond or a divalent group selected from the group consisting of:
   —R²—, —R³—, —R⁴—, —O—, —C(═O)—, —C(═O)O—, —OC(═O)—,

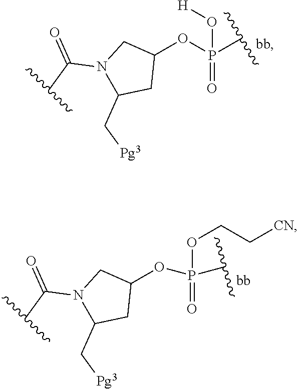

wherein Pg³ is H or Pg²;

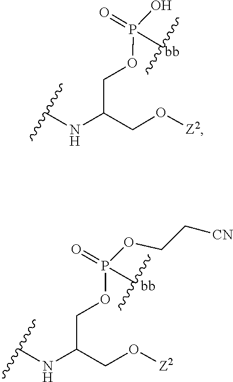

wherein Z² is an H or a hydroxyl protecting group; and

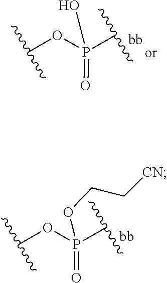

wherein bb represents the point of attachment to Oligo, and
Oligo is an oligonucleotide;

each R⁶ is independently selected from the group consisting of: H; C₁₋₃ alkyl; C₁₋₃ haloalkyl; and halo;

each R⁷ is independently selected from the group consisting of: H; and C₁₋₃ alkyl.

a and b are each independently selected integers from 1 to 10;

c and d are each independently selected integers from 0 to 10; and

each occurrence of R^{a} is independently selected from the group consisting of: halo, cyano, C₁₋₆ alkyl, C₁₋₆ haloalkyl, C₁₋₆ alkoxy, and C₁₋₆ haloalkoxy.

Variable R^{X}

In some embodiments, each R^{X} is selected from the group consisting of:
H; and

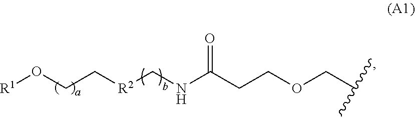

In some embodiments, each R^{X} is selected from the group consisting of:
—CH₂OR^{X2}, wherein R^{X2} is H, C₁₋₆ alkyl, or a hydroxyl protecting group; and

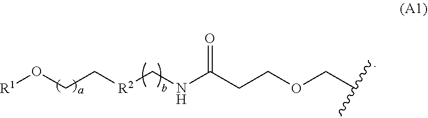

In certain embodiments, the hydroxyl protecting group is selected from the group consisting of: a silyl protecting group; 4-monomethoxytrityl (MMTR); 4,4-dimethoxytrityl (DMTR); and trityl.

In certain of these embodiments, the silyl protecting group is selected from the group consisting of: tert-butyldimethylsilyl (TBMDS); tert-butyldiphenylsilyl (TBDPS), and triisopropylsilyl (TIPS).

In some embodiments, at least two R^{X} are each independently a group of Formula (A1).

In certain of these embodiments, each R^{X} is independently a group of Formula (A1).

In some embodiments, each R^{X} is a ground of Formula (A1); and each R^{X} is the same.

Variable R¹

In some embodiments, each R¹ is an independently selected carbohydrate moiety. In certain of these embodiments, each R¹ is an independently monosaccharide or polysaccharide (e.g., monosaccharide or disaccharide). In certain embodiments, each R¹ is selected from the group consisting of: galactose, N-acetylgalactosamine, mannose, glucose, glucosamine and fucose. In certain of these embodiments, each R¹ is the same.

In certain embodiments, each R¹ is independently a group of Formula (B1) or (B2):

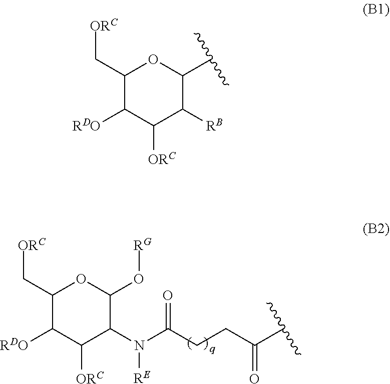

wherein:

R^{D} is selected from the group consisting of: R^{C} and

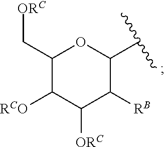

each R^{B} is independently selected from the group consisting of: —NR^{E}R^{F} and —OR^{C};

each R^{C} is independently selected from the group consisting of: H and C(═O)C₁₋₆ alkyl;

each R^{E} is independently C(═O)C₁₋₆ alkyl;

each R^{F} is independently selected from the group consisting of: H and

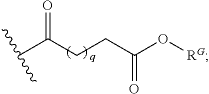

R^{G} is C₁₋₆ alkyl; and

each q is independently an integer selected from 1 to 10.

In certain of these embodiments, each R¹ is independently a group having Formula (B1-a), (B1-b), or (B2-a):

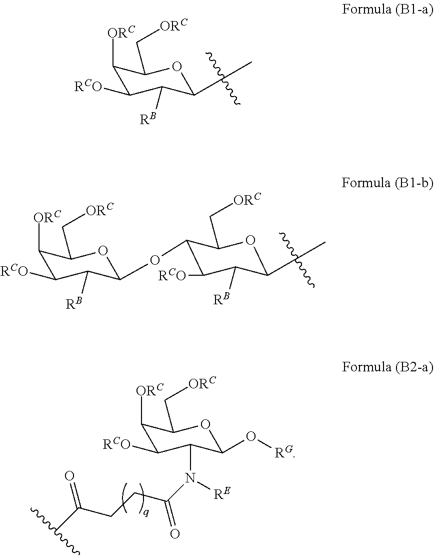

In certain embodiments of Formula (B1), (B2), (B1-a), (B1-b), or (B2-a), each R^{B} is independently NR^{E}R^{F}. In certain of these embodiments, each R^{E} is independently C(═O)C₁₋₆ alkyl. For example, each R^{E} can be C(═O)CH₃. In certain embodiments, each R^{F} is H. In certain embodiments, each R^{F} is

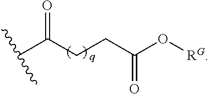

In certain embodiments of Formula (B1), (B2), (B1-a), (B1-b), or (B2-a), each R^{B} is NHC(═O)CH₃.

In certain embodiments of Formula (B1), (B2), (B1-a), (B1-b), or (B2-a), each R^{B} is

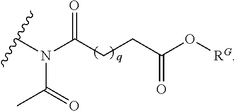

In certain embodiments of Formula (B1), (B2), (B1-a), (B1-b), or (B2-a), each R^{B} is independently —OR^{C}.

In certain embodiments of Formula (B1), (B2), (B1-a), (B1-b), or (B2-a), each R^{C} is independently C(═O)C₁₋₆ alkyl. For example, each R^{C} can be C(═O)CH₃.

In certain embodiments of Formula (B1), (B2), (B1-a), (B1-b), or (B2-a), each R^{C} is H.

In certain embodiments of Formula (B2) or (B2-a), each R^{G} is CH₃.

In certain embodiments, each R¹ is selected from the group consisting of the following:

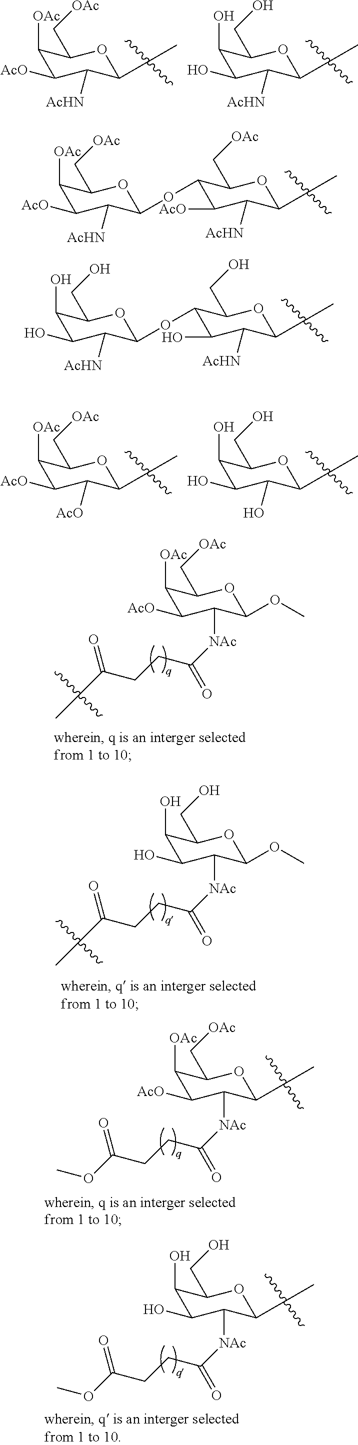

In some embodiments, each R¹ is the same.

For example, each R¹ can be

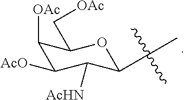 [SMILES: *C1OC(COC(C)=O)C(OC(C)=O)C(OC(C)=O)C1NC(C)=O]

As another non-limiting example, each R¹ can be

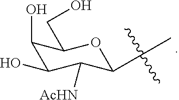 [SMILES: *C1OC(CO)C(O)C(O)C1NC(C)=O]

Variable R², a, and b

In some embodiments, each R² is independently *—C(═O)NR⁷— or *—NR⁷C(═O)—. In certain embodiments, each R² is independently *—C(═O)NR⁷—. In certain of these embodiments, each R² is *—C(═O)NH—.

In some embodiments, each R² is independently —C(R⁶)₂—. In certain of these embodiments, each R² is —CH₂—. In some embodiments, each R² is the same.

In some embodiments, each a is independently 1, 2, 3, or 4. In some embodiments, each a is independently 5, 6, or 7. In some embodiments, each a is independently 8, 9, or 10.

In some embodiments, each a is an independently selected integer from 1 to 4. In certain embodiments, each a is independently 2 or 3. In some embodiments, each a is the same.

In some embodiments, each b is independently 1, 2, 3, or 4. In some embodiments, each b is independently 5, 6, or 7. In some embodiments, each b is independently 8, 9, or 10.

In some embodiments, each b is an independently selected integer from 1 to 4. In certain of these embodiments, each b is independently 2 or 3. In some embodiments, each b is the same.

In certain embodiments, each a is the same; each b is the same; and each R² is the same.

In certain of these embodiments, a is an integer from 1 to 4; b is an integer from 1 to 4; and R² is *—C(═O)NR⁷. For example, a can be 3; b can be 3; and R² can be *—C(═O)NH.

In certain embodiments (when each a is the same; each b is the same; and each R² is the same), a is an integer from 1 to 4; b is an integer from 1 to 4; and R² is —C(R⁶)₂—.

For example, R² can be —CH₂—; and 3≤(a+b)≤5.

Variable R³

In some embodiments, R³ is

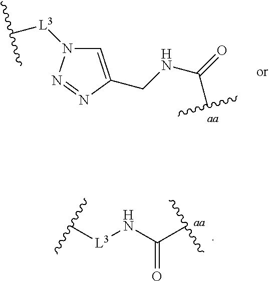

In certain embodiments, R³ is

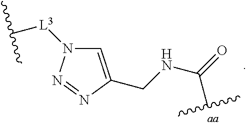

In certain embodiments, R³ is

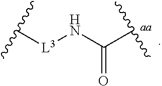

In some embodiments, L³ is —C(R⁶)₂—. In certain embodiments, L³ is —CH₂—.

Variable R⁴, c, and d

In some embodiments, c is independently 0 or 1. In some embodiments, c is independently 2, 3, or 4. In some embodiments, c is independently 5, 6, or 7. In some embodiments, c is independently 8, 9, or 10.

In some embodiments, c is an integer from 1 to 2.

In some embodiments, c is an integer from 2 to 5.

In some embodiments, c is an integer from 3 to 7.

In some embodiments, R⁴ is *—C(═O)NR⁷—. In certain of these embodiments, R⁴ is *—C(═O)NH—.

In some embodiments, R⁴ is —C(R⁶)₂—. In certain of these embodiments, R⁴ is —CH₂—.

In some embodiments, d is independently 0 or 1. In some embodiments, d is independently 2, 3, or 4. In some embodiments, d is independently 5, 6, or 7. In some embodiments, d is independently 8, 9, or 10.

In some embodiments, d is an integer from 1 to 2.

In some embodiments, d is an integer from 3 to 7.

In certain embodiments, R⁴ is —C(R⁶)₂—; and each of c and d is independently 1 or 2. In certain of these embodiments, R⁴ is —CH₂—; and each of c and d is 1.

In certain embodiments, R⁴ is —C(R⁶)₂—; and 4≤(c+d)≤12. In certain of these embodiments, R⁴ is —CH₂—; and 7≤(c+d)≤10.

In certain embodiments, R⁴ is *—C(═O)NR⁷—; and 5≤(c+d)≤10. In certain of these embodiments, R⁴ is *—C(═O)NR⁷—; and 7≤(c+d)≤9. In certain of the foregoing embodiments, c is 3.

Non-Limiting Combinations

In certain embodiments, the compound of Formula (I) is selected from the group consisting of the following:

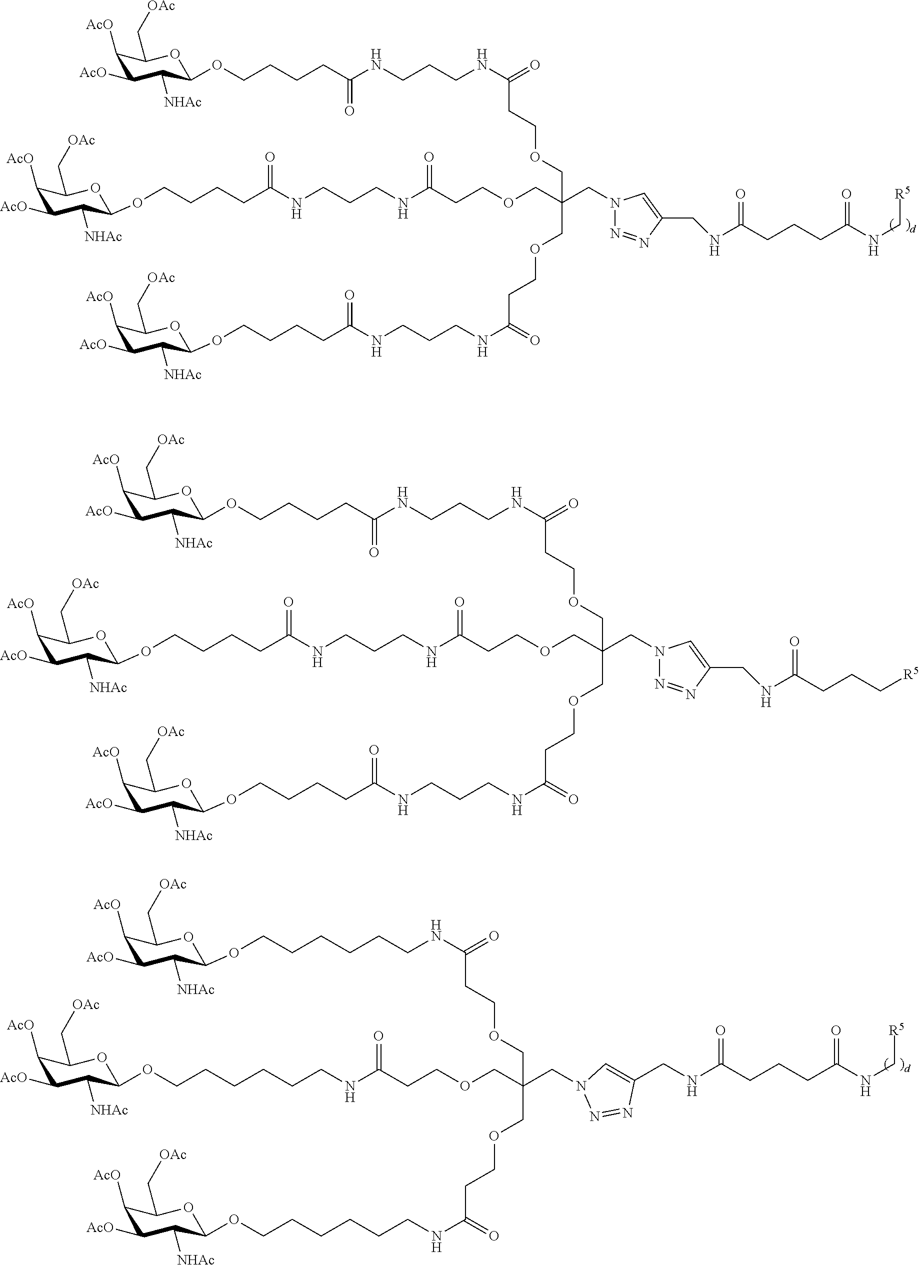

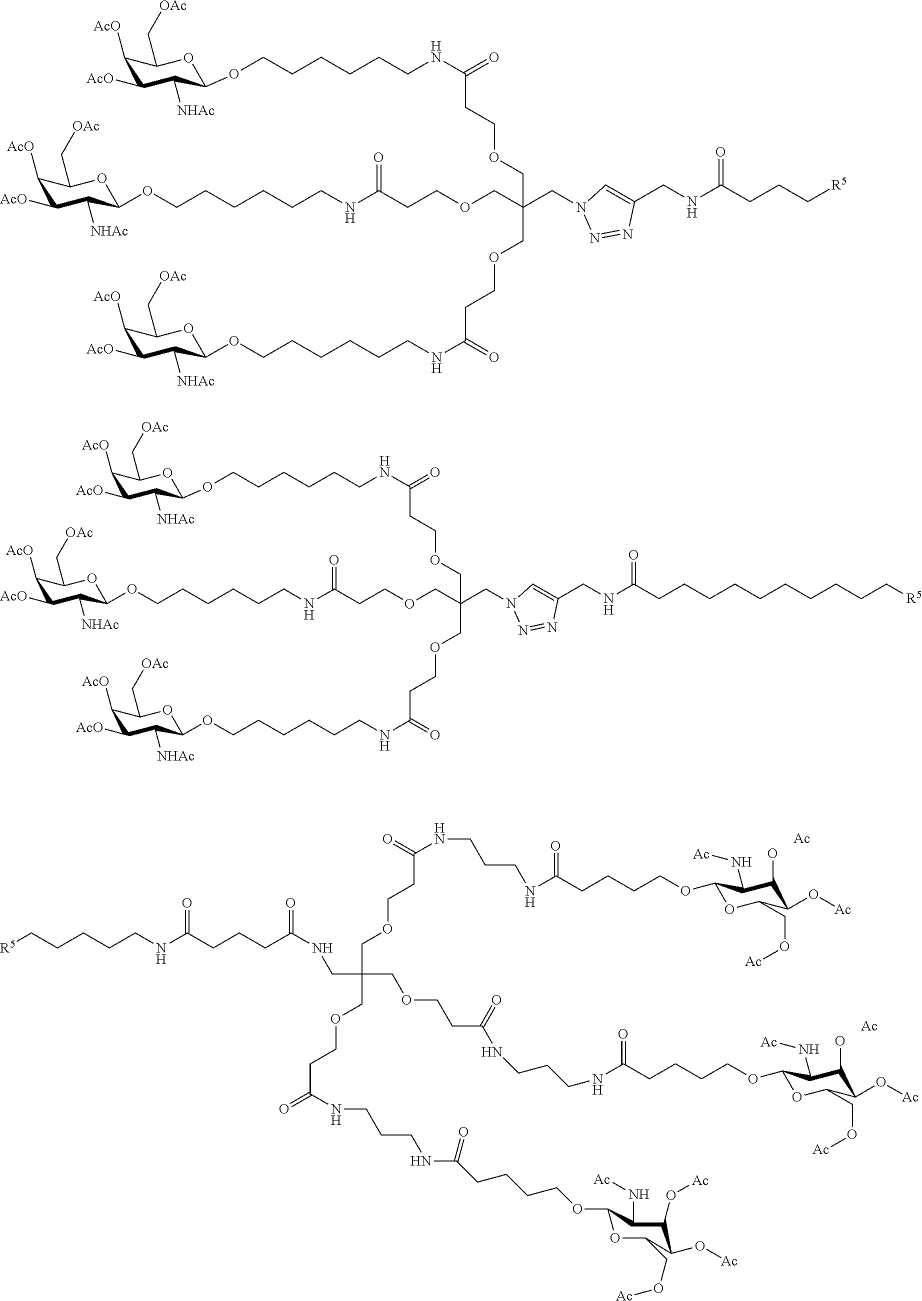

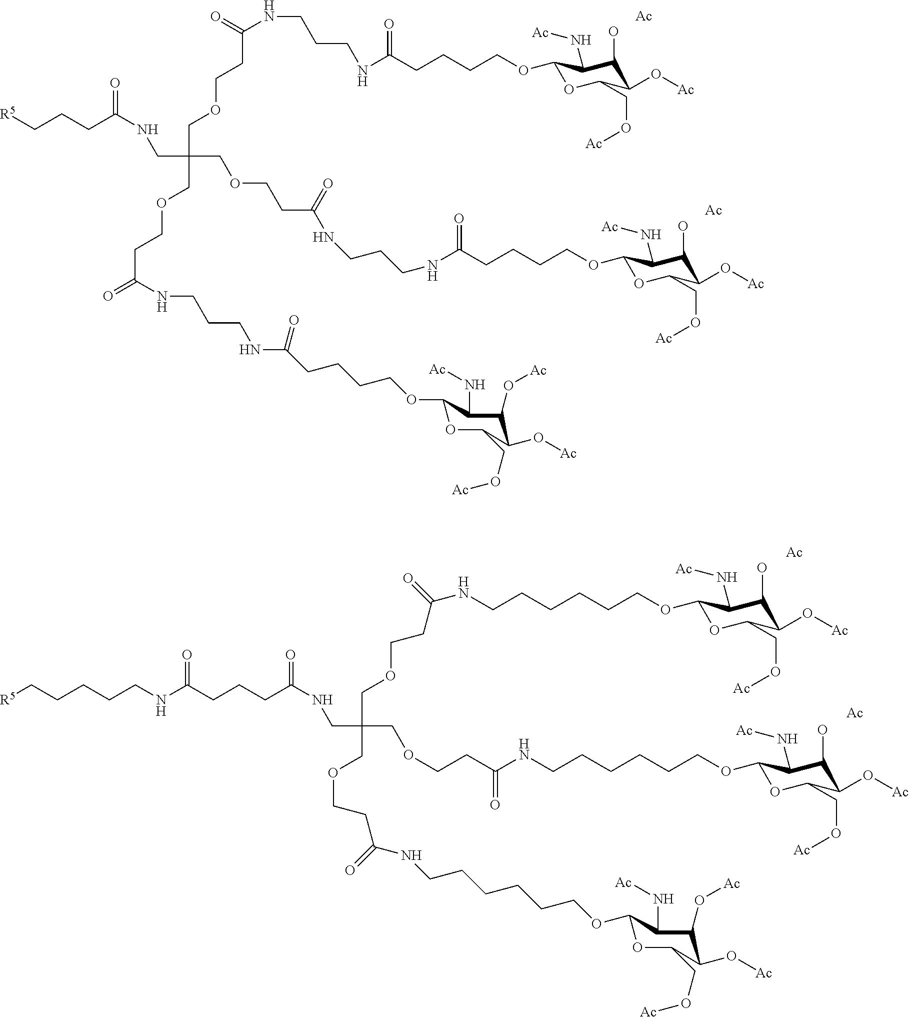

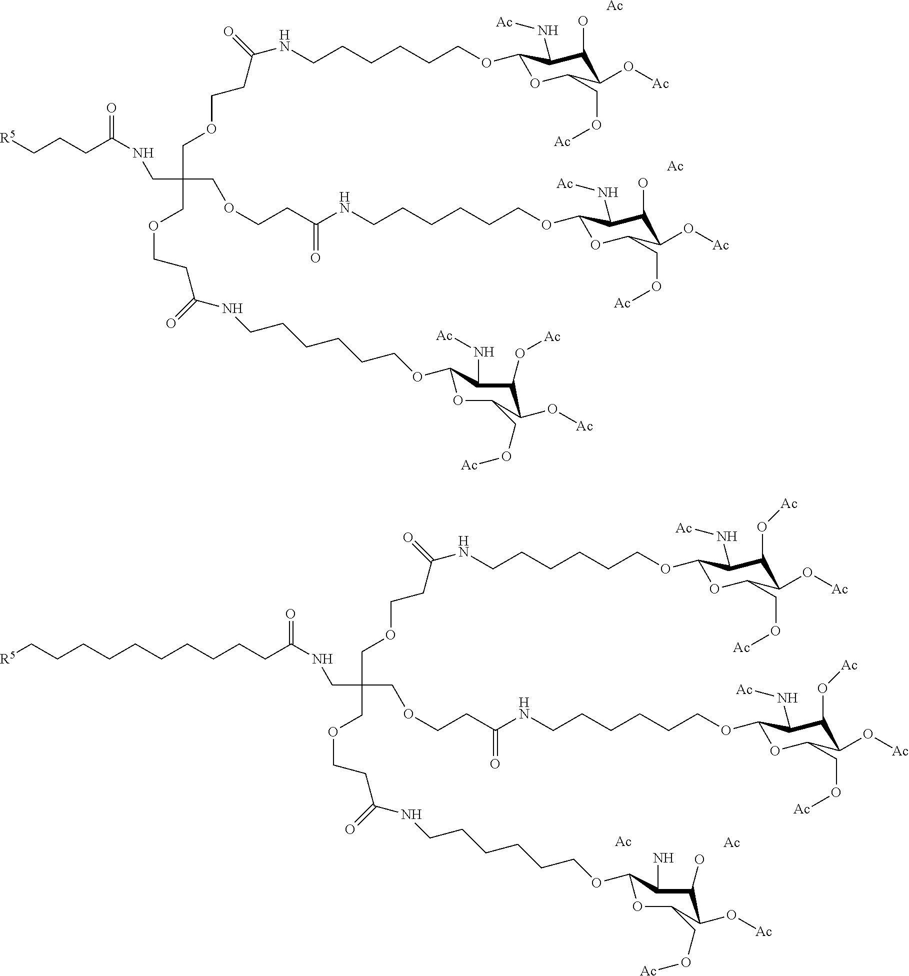

Variable R⁵

In some embodiments, R⁵ is C(O)OH or

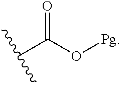

In certain embodiments, R⁵ is C(O)OH.

In certain embodiments, R⁵ is

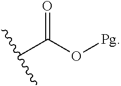

In certain embodiments, Pg is a carboxyl activating group. As used herein, a “carboxyl activating group” is a chemical moiety that, upon covalent bonding with a carboxyl oxygen atom, converts said oxygen atom into a leaving group. Accordingly, when Pg is a carboxyl activating group, the “—O-Pg” group in

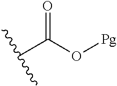

is a leaving group. Non-limiting examples of carboxyl activating groups include N-centered heterocyclyl (e.g., succinimidyl) and electron-deficient heteroaryl and aryl (e.g., pentafluorophenyl).

In certain embodiments, Pg is

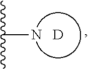

wherein Ring D is a 5-10 membered heteroaryl or 4-10 membered heterocyclyl, each optionally substituted with 1-6 substituents each independently selected from the group consisting of: halo, oxo, NO₂, C(O)C₁₋₄ alkyl, C(O)OC₁₋₄ alkyl, S(O)C₁₋₄ alkyl, C₁₋₆ alkyl, C₁₋₆ haloalkyl, and —OH. For example, Pg can be

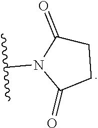 [SMILES: *N1C(=O)CCC1=O]

As another non-limiting example, Pg can be

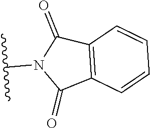 [SMILES: *N1C(=O)c2ccccc2C1=O]

which is optionally substituted with 1-6 substituents each independently selected from the group consisting of: halo, NO₂, C(O)C₁₋₄ alkyl, C(O)OC₁₋₄ alkyl, S(O)C₁₋₄ alkyl, C₁₋₆ alkyl, C₁₋₆ haloalkyl, and —OH (e.g., unsubstituted or substituted with 1-6 independently selected halo).

In certain embodiments, Pg is C₆₋₁₀ aryl or 5-10 membered heteroaryl substituted with 1-6 substituents each independently selected from the group consisting of: —F; —Cl; —NO₂; C(O)C₁₋₄ alkyl, C(O)OC₁₋₄ alkyl, and S(O)C₁₋₄ alkyl.

In certain of these embodiments, Pg is

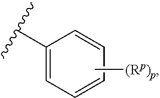

wherein p is an integer from 1 to 5; and each R^{p} is —F, —Cl, or —NO₂. In certain embodiments, each R^{p} is —F. As a non-limiting example of the foregoing embodiments, Pg can be

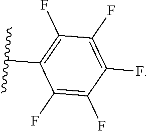 [SMILES: *c1c(F)c(F)c(F)c(F)c1F]

In certain embodiments, R⁵ is

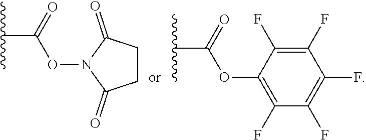

In some embodiments, R⁵ is

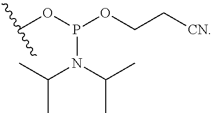 [SMILES: *OP(OCCC#N)N(C(C)C)C(C)C]

In some embodiments, R⁵ is

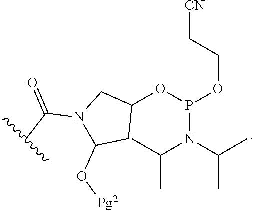

In certain embodiments, R⁵ is

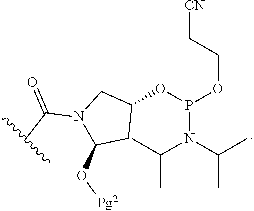

In some embodiments, Pg² and Z are each independently selected from the group consisting of: a silyl protecting group; 4-monomethoxytrityl (MMTR); 4,4-dimethoxytrityl (DMTR); and trityl.

In certain of these embodiments, the silyl protecting group is selected from the group consisting of: tert-butyldimethylsilyl (TBMDS); tert-butyldiphenylsilyl (TBDPS), and triisopropylsilyl (TIPS).

Non-Limiting Exemplary Compounds

In certain embodiments, the compound is selected from the group consisting of compounds GalNAc-1 through GalNAc-12. For example, the compound can be selected from the group consisting of compounds GalNAc-1 through GalNAc-10.

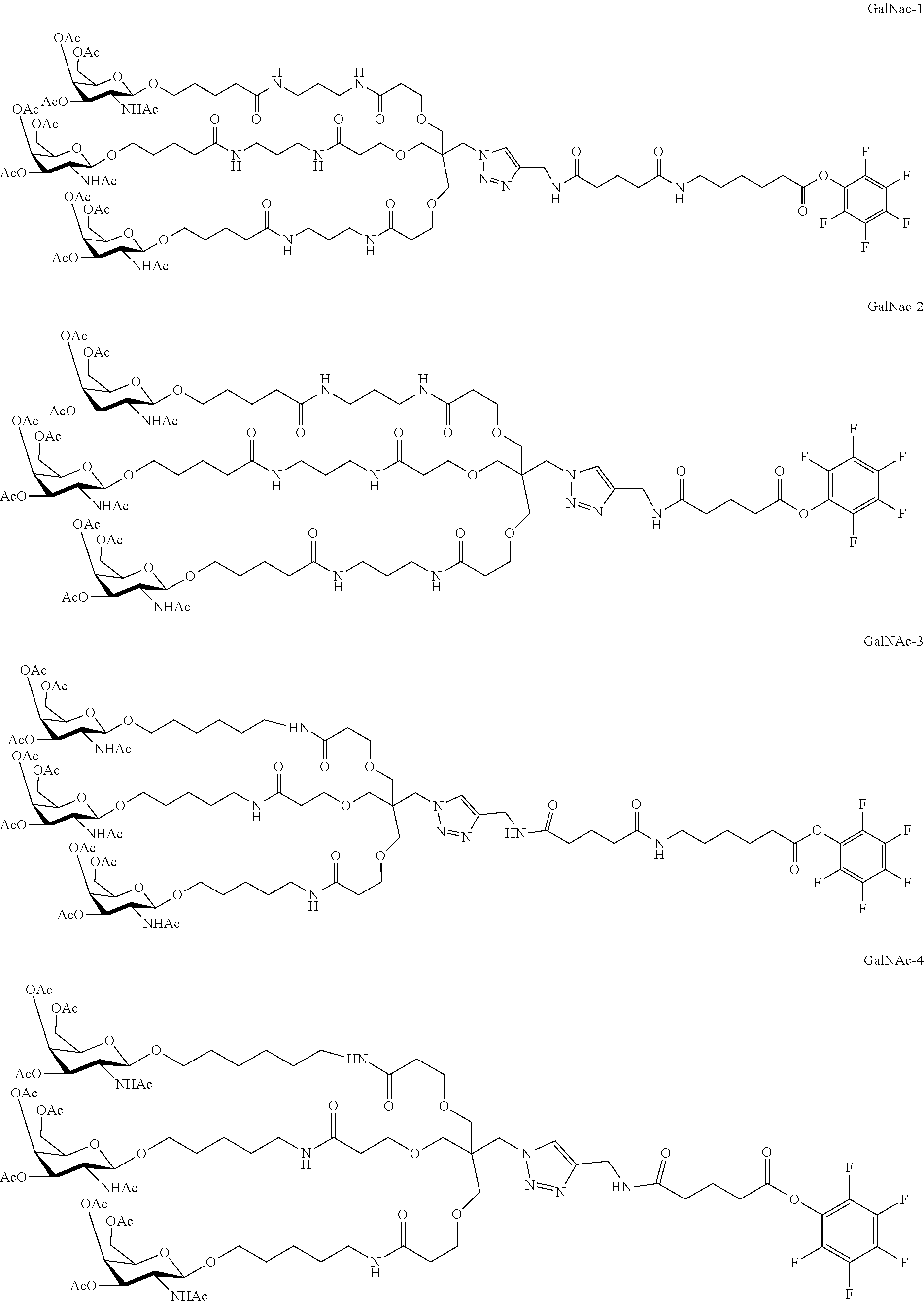

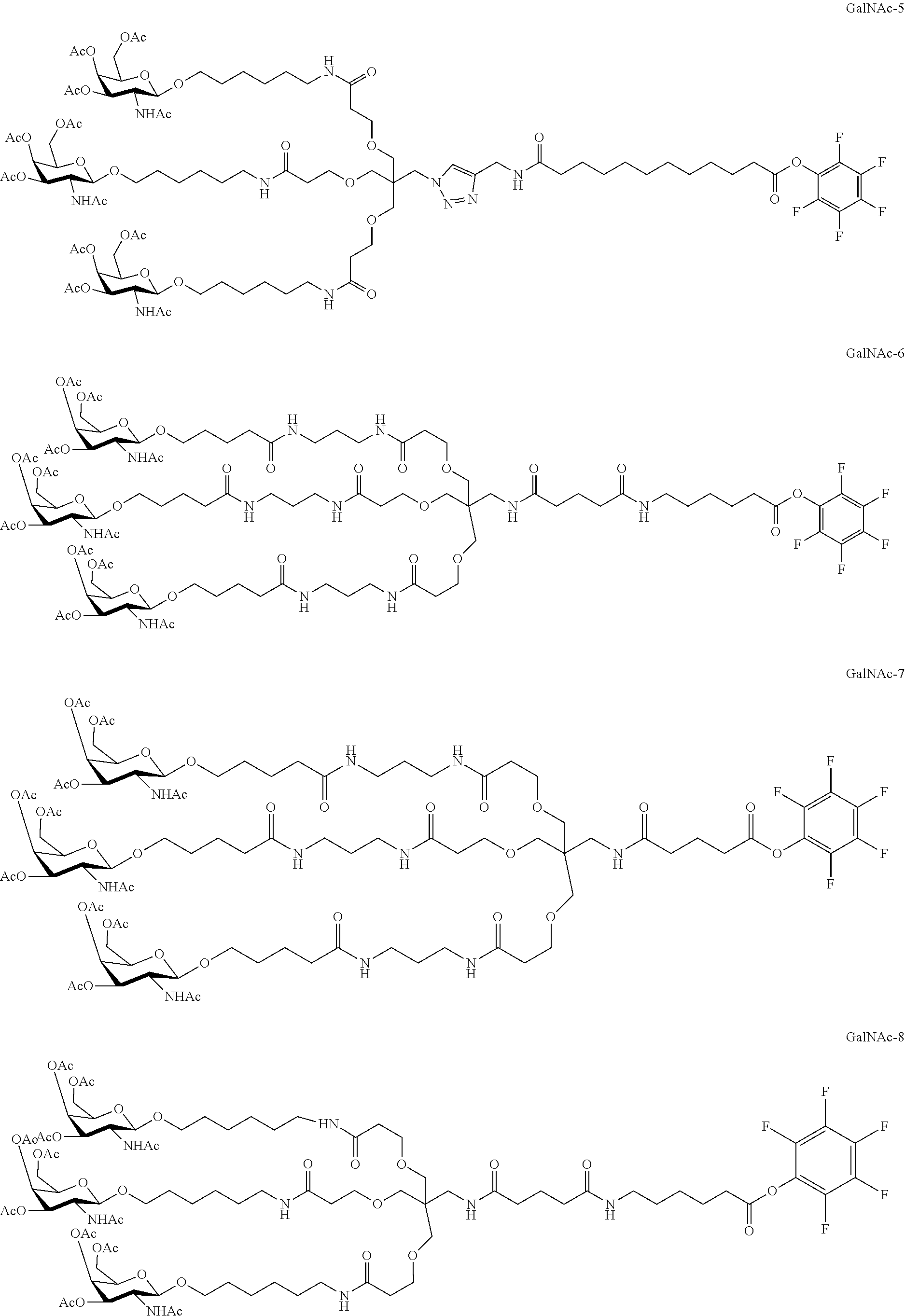

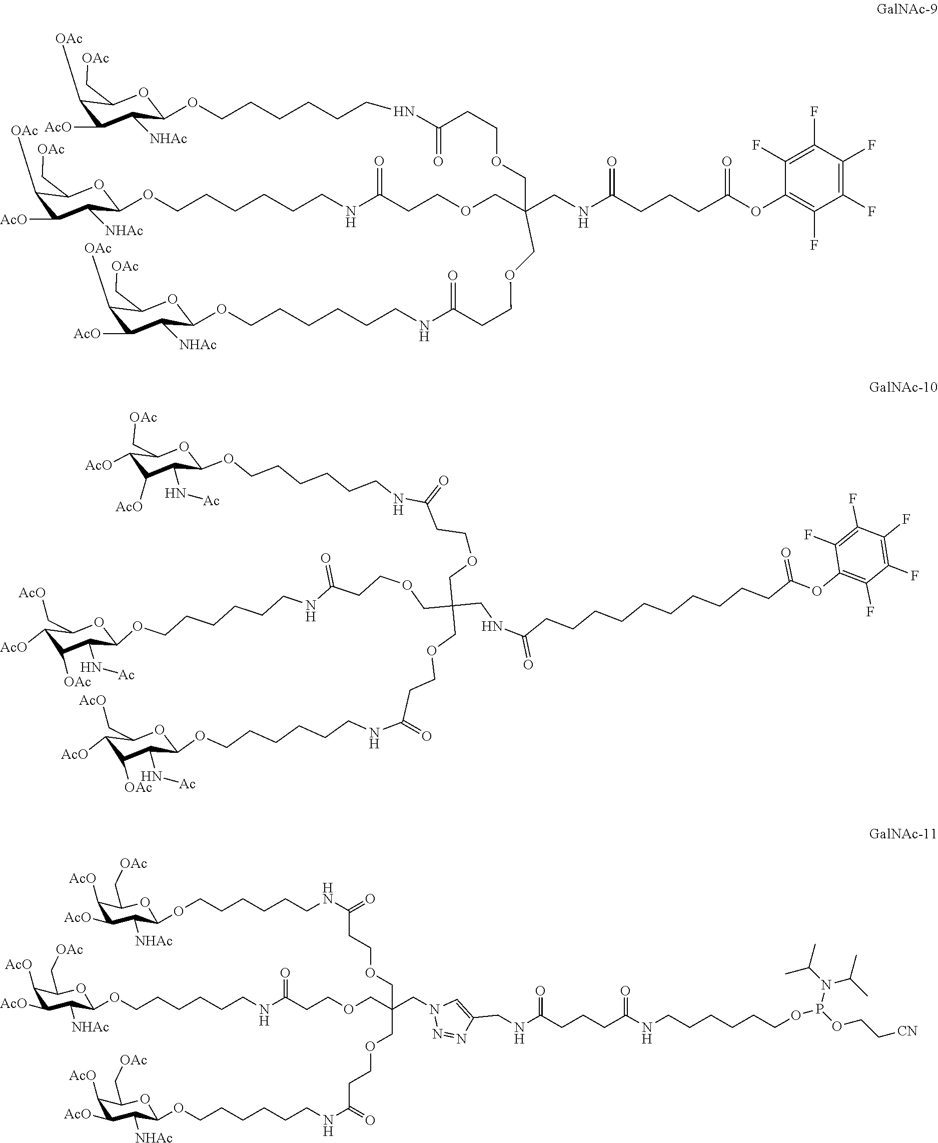

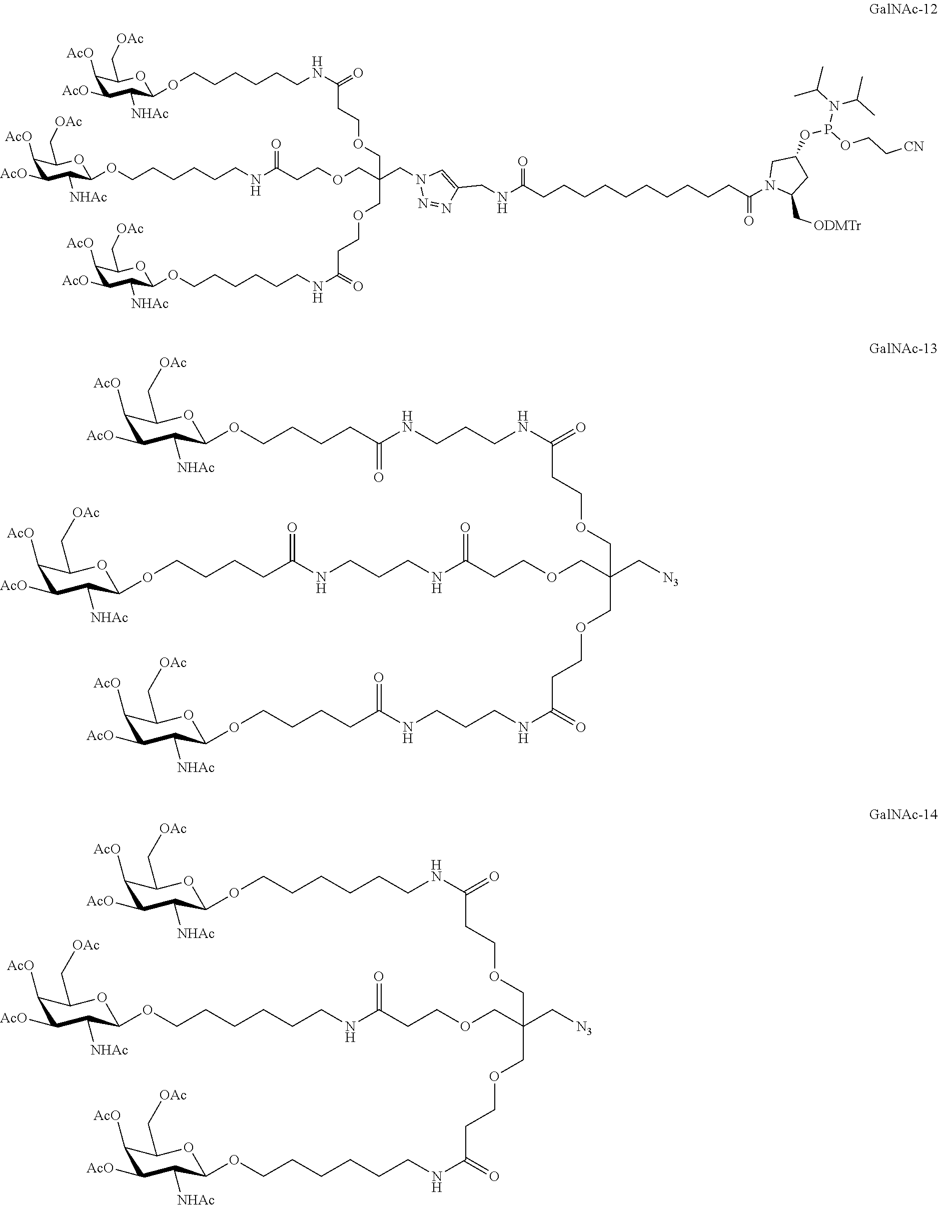

GalNAc-13 and GalNAc-14 are useful e.g., as intermediates in the preparation of Formula (I) compounds.

Conjugate Compounds

In some embodiments, the compound of Formula (I) is a conjugate compound of Formula (II):

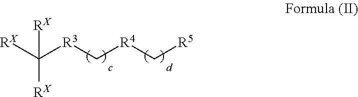

or a pharmaceutically acceptable salt thereof, wherein:

R⁵ is

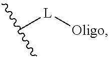

wherein Oligo is an oligonucleotide; and

-L-, R^{X} (including R¹, R², a, and b), R³, c, R⁴, and d can be as defined for Formula (I) anywhere herein.

In some embodiments, Oligo is an oligonucleotide that is attached to L via the 5′-end, 3′-end, or sequence middle of any strand via a phosphate group.

Variable L

In some embodiments, L is a bond.

In some embodiments, L is C(═O).

In some embodiments, L is —O—.

In some embodiments, L is

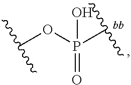

wherein bb is the point of attachment to Oligo.

In some embodiments, L is selected from the group consisting of:

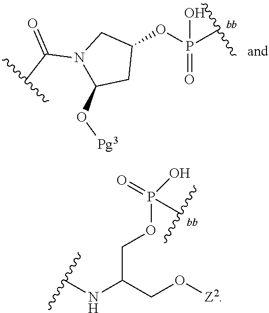

In certain of these embodiments, L is

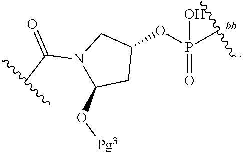

In certain embodiments, Pg³ is H. In certain embodiments, Pg³ is a hydroxyl protecting group.

In certain embodiments, L is

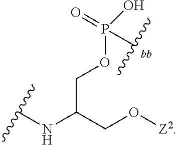

In certain embodiments, Z² is H. In certain embodiments, Z² is a hydroxyl protecting group.

In certain embodiments, the hydroxyl protecting group is selected from the group consisting of: a silyl protecting group; 4-monomethoxytrityl (MMTR); 4,4-dimethoxytrityl (DMTR); and trityl.

In certain of these embodiments, the silyl protecting group is selected from the group consisting of: tert-butyldimethylsilyl (TBMDS); tert-butyldiphenylsilyl (TBDPS), and triisopropylsilyl (TIPS).

Oligonucleotide Moiety

In some embodiments, Oligo is an oligonucleotide that comprises a single-stranded oligonucleotide and/or a double-stranded oligonucleotide.

In certain of these embodiments, Oligo is a single-stranded oligonucleotide.

In certain embodiments, Oligo is a double-stranded oligonucleotide.

In some embodiments, the oligonucleotide is selected from the group consisting of: DNA, siRNA, miRNA, pre-miRNA, antagomir, mRNA, antisense oligonucleotide (ASO), Aptamer, crRNA, tracRNA, and sgRNA.

The oligonucleotide herein can comprise unmodified nucleotides and/or modified nucleotides. Non-limiting examples of modified nucleotides include: 2′-O-(2-methoxyethyl)-modified nucleotides; 2′-O-alkyl modified nucleotides (e.g., 2′-O-methyl modified nucleotides); 2′-O-allyl modified nucleotides; 2′-C-allyl modified nucleotides; 2′-fluoro modified nucleotides; 2′-deoxy modified nucleotides; 2′-hydroxy modified nucleotides; locked nucleic acids (LNAs) modified nucleotides; hexitol nucleic acids (HNAs) modified nucleotides; glycol nucleic acids (GNAs) modified nucleotides, and unlocked nucleic acid (UNAs) modified nucleotides. In certain embodiments, the modified nucleotide is selected from the group consisting of: 2′-O-alkyl modified nucleotides and 2′-fluoro modified nucleotides.

In some embodiments, the oligonucleotide comprises a modifying group, wherein the modifying group is selected from the group consisting of: cholesterol, polyethylene glycol, fluorescent probes, biotin, polypeptides, vitamins, tissue targeting molecules, and a combination thereof. In certain of these embodiments, the modifying group is a terminal modifying group.

Oligo can be attached to L via the 5′-end, 3′-end or sequence middle of any strand via a phosphate group. In certain embodiments, the phosphate group is a phosphodiester group. In certain embodiments, the phosphate group is a modified phosphate group. In certain of these embodiments, the modified phosphate group is selected from one or more of: thio modified phosphate (e.g., phosphorothioate), and amino modified phosphate.

In some embodiments, Oligo comprises one or more peptide nucleic acids and/or morpholino nucleic acids (e.g., morpholino antisense oligonucleotides).

In some embodiments, the Oligo is an oligonucleotide of from 5 to 100 base pairs. For example, Oligo can be an oligonucleotide of 5 to 10, 10 to 20, 20 to 30, 30 to 40, 40 to 50, 50 to 60, 60 to 70, 70 to 80, 80 to 90, or 90 to 100 base pairs.

In some embodiments, the compound of Formula (II) is synthesized via solid-phase synthesis or liquid-phase synthesis. In certain embodiments, the compound of Formula (II) is synthesized via solid-phase synthesis. In certain embodiments, the compound of Formula (II) is synthesized via liquid-phase synthesis.

In some embodiments, the oligonucleotide can be as described anywhere in paragraphs [0335]-[410] in US 2015/0119444, or paragraphs [0341]-[416] in US 2015/0119445, and these sections are incorporated herein by reference.

Non-Limiting Examples of Conjugate Compounds

Non-limiting examples of Formula (II) compounds include the following (wherein n, m, and m′ are each independently selected integers from 1 to 10):

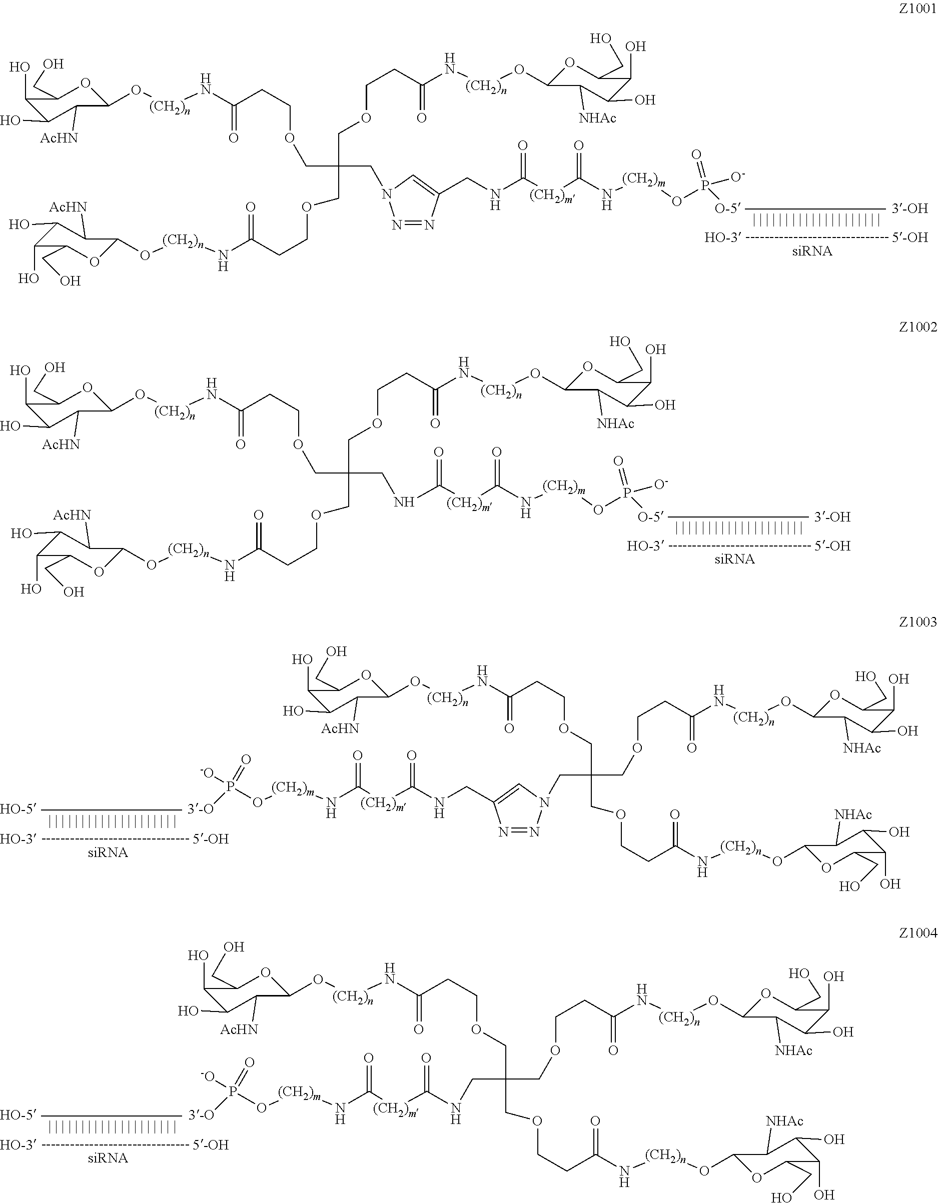

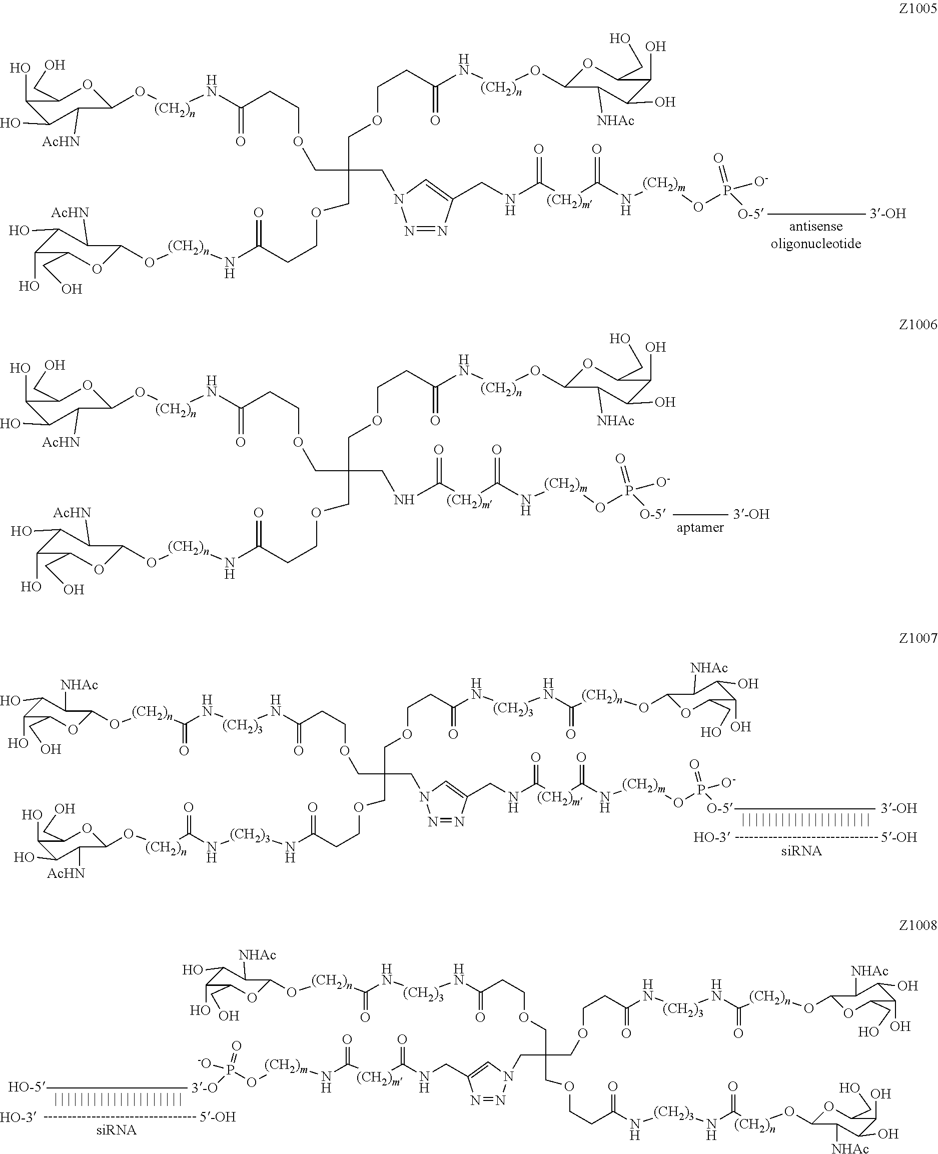

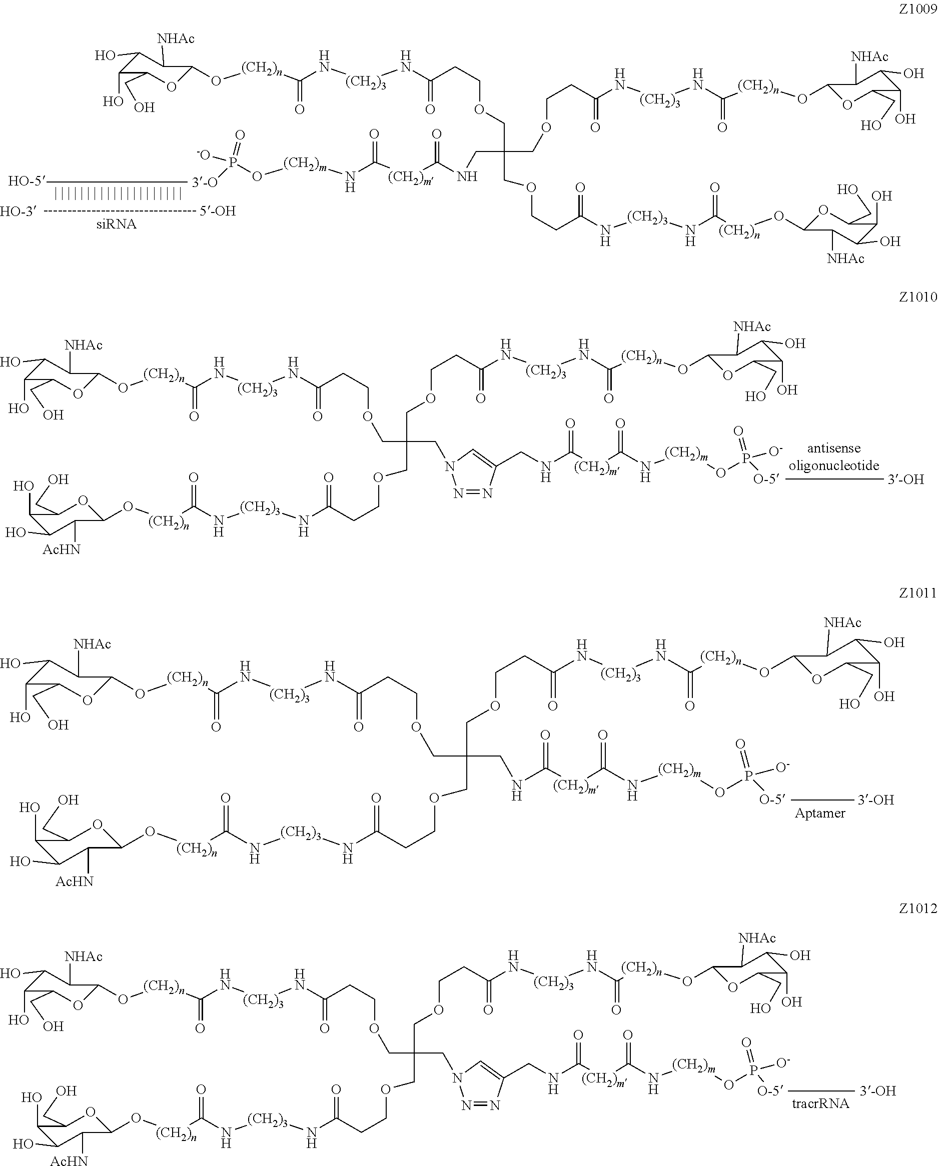

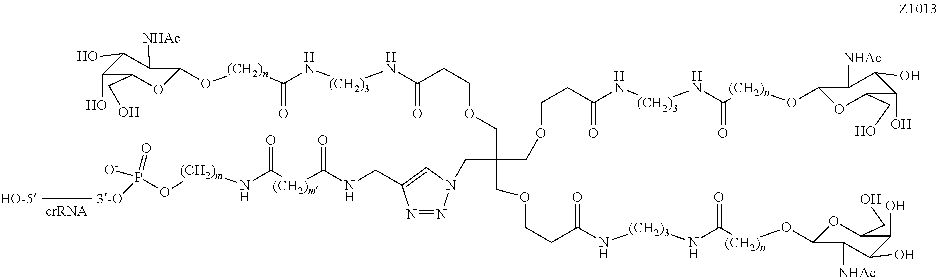

Pharmaceutical Compositions and Administration

General

In another aspect, this disclosure features pharmaceutical compositions comprising a chemical entity as described herein (e.g., a compound of Formula (II) or a pharmaceutically acceptable salt thereof) and a pharmaceutically acceptable excipient.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%.

Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, the pharmaceutical composition is formulated in a dosage form selected from the group consisting of: powders, tablets, granules, capsules, solutions, emulsions, suspensions, injections, sprays, aerosols, dry powder inhalations, and microneedle patches.

In some embodiments, the pharmaceutical composition is suitable for administration to a subject in need thereof intravenously, intramuscularly, subcutaneously, via microneedle patches, orally, via oral or nasal spray, or topically.

In some embodiments, the subject is a mammal, including bovine, equine, sheep, swine, canine, feline, rodent, and primate. For example, the subject can be human.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, a unit dose is less than 1.4 mg per kg of bodyweight of the subject, or less than 10, 5, 2, 1, 0.5, 0.1, 0.05, 0.01, 0.005, 0.001, 0.0005, 0.0001, 0.00005 or 0.00001 mg per kg of bodyweight. In some embodiments, a unit dose is from 0.00001 to 10 mg per kg of bodyweight. In some embodiments, a unit dose is from 0.001 to 2 mg per kg of bodyweight.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

Methods of Treatment

In another aspect, this disclosure features methods for treating and/or preventing pathological conditions or diseases in a subject, wherein the conditions or diseases are caused by the expression of one or more genes in liver cells, the method comprising administering to the subject a chemical entity as described herein (e.g., a compound of Formula (II) or a pharmaceutically acceptable salt thereof), or a pharmaceutical composition comprising a chemical entity as described herein (e.g., a compound of Formula (II) or a pharmaceutically acceptable salt thereof), and a pharmaceutically acceptable excipient.

In some embodiments, the one or more genes are selected from: HBV genome, HCV genome, PCSK9, xanthine oxidase, URAT1, APOB, liver fibrosis-related genes (AP3S2, AQP2, AZIN1, DEGS1, STXBP5L, TLR4, TRPM5, etc.), and genes related to non-alcoholic fatty liver disease (PNPLA3, FDFT1), primary biliary cirrhosis (HLA-DQB1, IL-12, IL-12RB2, etc.).

In some embodiments, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, hepatic porphyrins.

In some embodiments, the subject is a mammal, including bovine, equine, sheep, swine, canine, feline, rodent, and primate. For example, the subject can be human.

In a further aspect, this disclosure features methods for detecting or localizing RNA in the liver of a subject, comprising administering to the subject a chemical entity as described herein (e.g., a compound of Formula (II) or a pharmaceutically acceptable salt thereof); or a pharmaceutical composition comprising a chemical entity as described herein (e.g., a compound of Formula (II) or a pharmaceutically acceptable salt thereof), and a pharmaceutically acceptable excipient.

In some embodiments, the subject is diagnosed with one or more conditions or diseases that are caused by the expression of one or more genes in liver cells

In certain embodiments, the one or more genes are selected from: HBV genome, HCV genome, PCSK9, xanthine oxidase, URAT1, APOB, liver fibrosis-related genes (AP3S2, AQP2, AZIN1, DEGS1, STXBP5L, TLR4, TRPM5, etc.), and genes related to non-alcoholic fatty liver disease (PNPLA3, FDFT1), primary biliary cirrhosis (HLA-DQB1, IL-12, IL-12RB2, etc.).

In certain embodiments, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, hepatic porphyrins.

In some embodiments, the subject is a mammal, including bovine, equine, sheep, swine, canine, feline, rodent, and primate. For example, the subject can be human.

In some embodiments, the chemical entity described herein (e.g., a compound of Formula (II) or a pharmaceutically acceptable salt thereof) is administered intravenously, intramuscularly, subcutaneously, via microneedle patches, orally, via oral or nasal spray, or topically.

In another aspect, provided herein is a compound (e.g., a compound of Formula (II)), or a pharmaceutically acceptable salt thereof, or a pharmaceutical composition thereof, for use in the treatment of conditions or diseases are caused by the expression of one or more genes in liver cells in a subject in need thereof. In some embodiments, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, hepatic porphyrins. The subject can be as defined anywhere herein. In some embodiments, the subject is a mammal (e.g., human).

In another aspect, provided herein is a compound (e.g., a compound of Formula (II)), or a pharmaceutically acceptable salt thereof, or a pharmaceutical composition thereof, for use in detecting or localizing RNA in the liver of a subject. The subject can be as defined anywhere herein. In some embodiments, the subject is a mammal (e.g., human).

In another aspect, provided herein is a use for a compound (e.g., a compound of Formula (II)), or a pharmaceutically acceptable salt thereof, or a pharmaceutical composition thereof, in the treatment of conditions or diseases are caused by the expression of one or more genes in liver cells in a subject in need thereof. In some embodiments, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, hepatic porphyrins. The subject can be as defined anywhere herein. In some embodiments, the subject is a mammal (e.g., human).

In another aspect, provided herein is a use of a compound (e.g., a compound of Formula (II)), or a pharmaceutically acceptable salt thereof, or a pharmaceutical composition thereof, in detecting or localizing RNA in the liver of a subject. The subject can be as defined anywhere herein. In some embodiments, the subject is a mammal (e.g., human).

In another aspect, provided herein is a use for a compound (e.g., a compound of Formula (II)), or a pharmaceutically acceptable salt thereof, or a pharmaceutical composition thereof, in the manufacture of a medicament for the treatment of conditions or diseases are caused by the expression of one or more genes in liver cells in a subject in need thereof. In some embodiments, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, hepatic porphyrins. The subject can be as defined anywhere herein. In some embodiments, the subject is a mammal (e.g., human).

In another aspect, provided herein is a use of a compound (e.g., a compound of Formula (II)), or a pharmaceutically acceptable salt thereof, or a pharmaceutical composition thereof, in the manufacture of a medicament for detecting or localizing RNA in the liver of a subject. The subject can be as defined anywhere herein. In some embodiments, the subject is a mammal (e.g., human).

Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

### EXAMPLES

The disclosure is further described in the following examples, which do not limit the scope of the disclosure as described in the claims. Specific conditions which are not noted in the examples are carried out under conventional conditions or conditions recommended by the manufacturer. The reagents or instruments used of which the manufacturer are not noted are conventional products commercially available.

### Example 1. Preparation of GalNAc-1

Synthetic Scheme

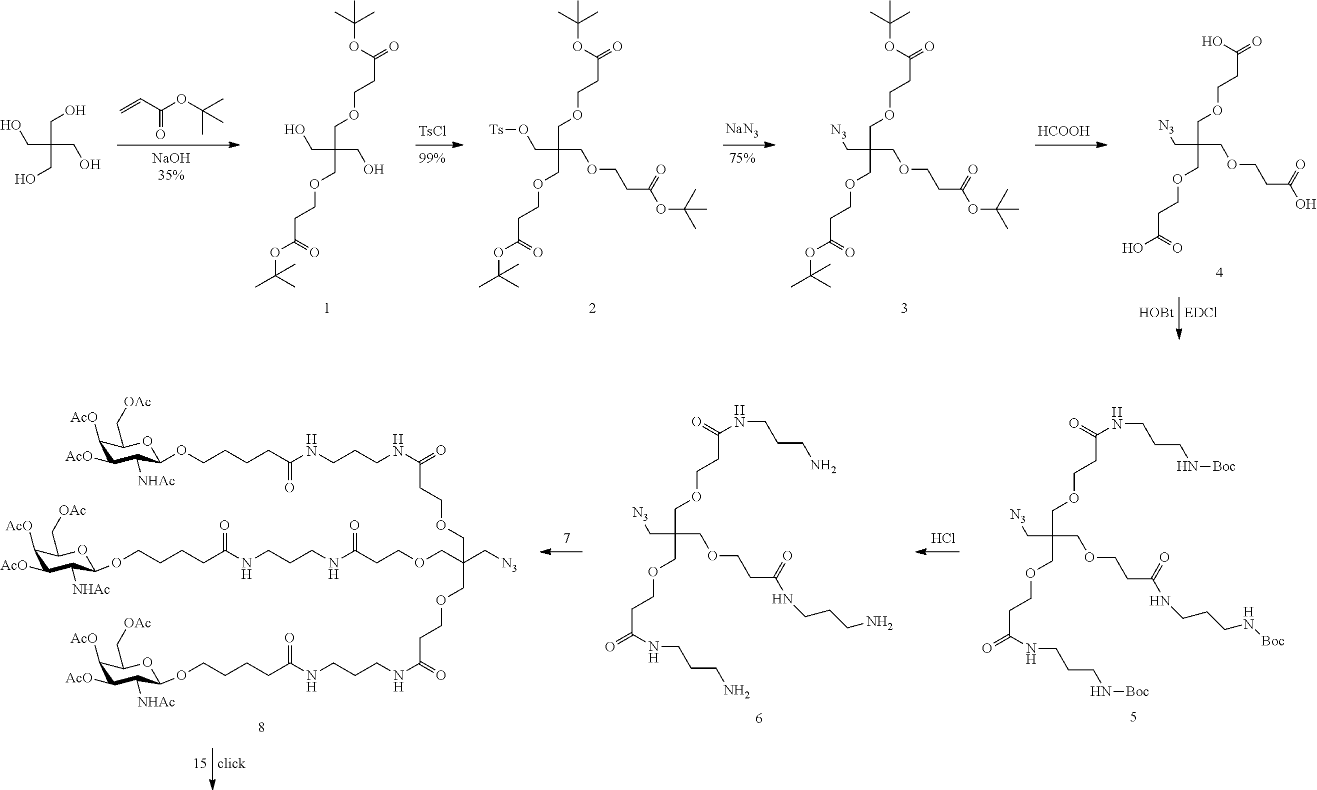

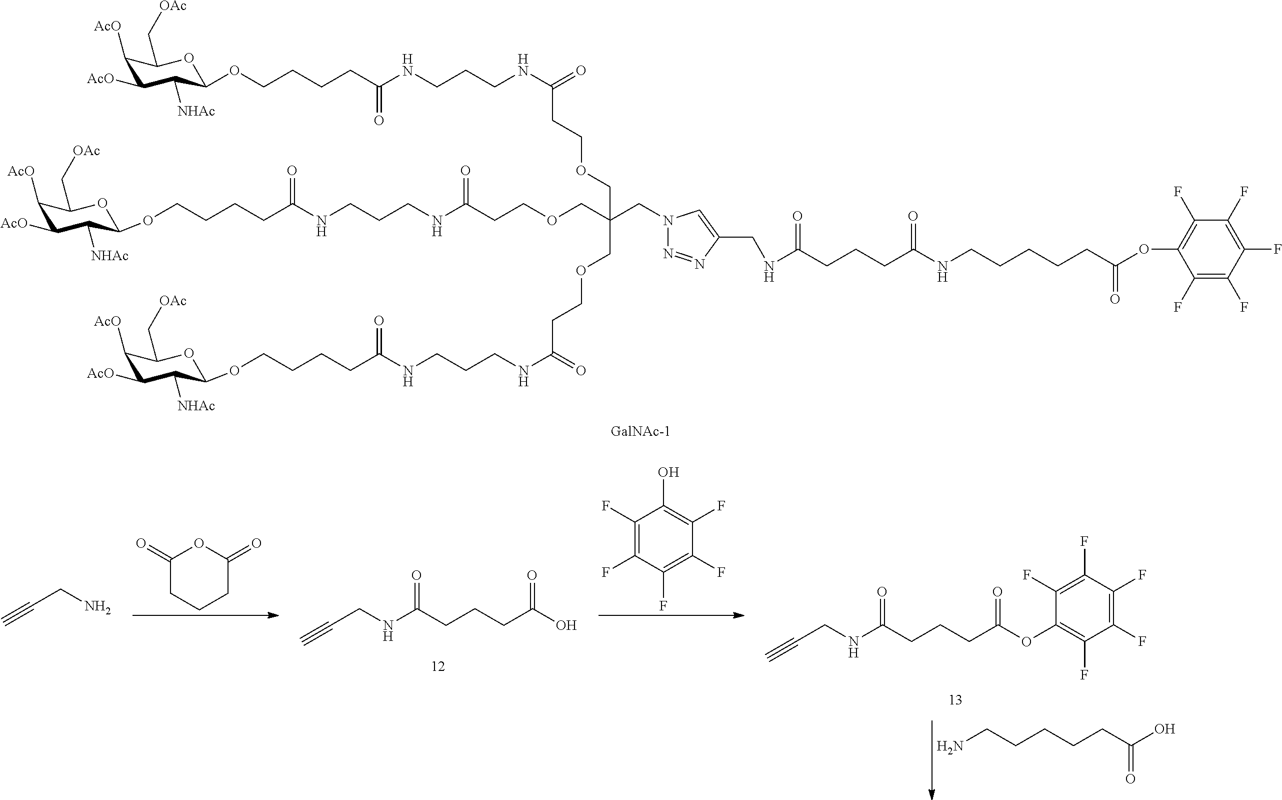

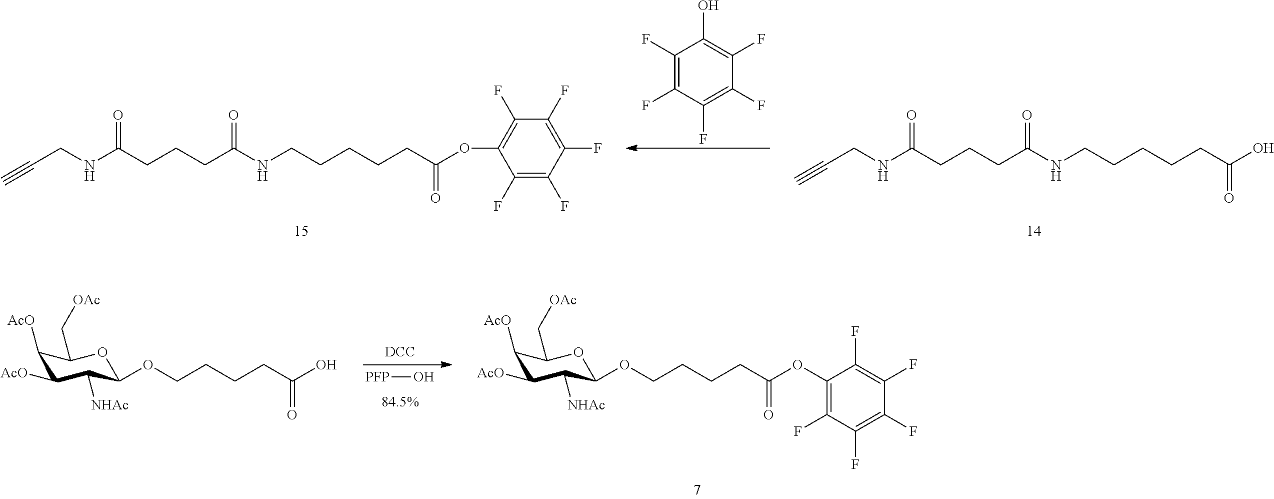

Procedure

(1) Pentaerythritol (50 g), NaOH solution (6 mL, aq., 50%), and tetrabutylammonium hydroxide (9.5 g) were added to tert-butyl acrylate (150 g) and the reaction mixture was then stirred at room temperature until the reaction was completed. Upon completion, ethyl acetate was added to extract the organic phase, which was then concentrated to yield the crude product. After column separation and purification (PE:EA=20:1-7:1) of the crude product, compound 1 was obtained;

(2) Compound 1 (10 g) and TsCl (6.5 g) were dissolved in pyridine (100 mL), and the resulting mixture was stirred at 70° C. Upon completion, the reaction mixture was concentrated to remove pyridine. Ethyl acetate was added to the residue, which is further concentrated and dried. After column separation and purification of the crude product (PE:EA=20:1-10:1), compound 2 was obtained;

(3) compound 2 (2 g) was dissolved in anhydrous DMF (50 mL), and then sodium azide (0.53 g) was added and the reaction mixture was heated to 100° C. Upon completion, the reaction mixture was cooled to room temperature and concentrated to remove DMF. Ethyl acetate was added to the residue, which is further concentrated and dried. After column separation and purification of the crude product (PE:EA=20:1-10:1), compound 3 was obtained;

(4) Compound 3 (2 g) was dissolved in formic acid (10 mL) and the reaction mixture was stirred at room temperature. Upon completion, the reaction mixture was concentrated to dryness and compound 4 was obtained;

(5) Compound 4 (1.6 g) was dissolved in dichloromethane (50 ml), then HOBt (2.23 g), EDCl (3.0 g), DIEA (3.3 g), and N-boc-1,3-diaminopropane (2.7 g) were added to the mixture. The reaction was stirred at room temperature until it was completed. Upon completion, dichloromethane and water were added to the reaction for extraction, after which the organic phase was dried and concentrated to dryness and yielded a crude product. After column separation and purification (dichloromethane:methanol=2%-5%) of the crude product, compound 5 was obtained;

(6) Compound 5 (2.5 g) was dissolved in dichloromethane (50 mL), then trifluoroacetic acid (20 mL, 2N) was added and the reaction was stirred at room temperature before it was completed. Upon completion, the reaction mixture was concentrated to obtain compound 6;

(7) 5-(((2R,3R,4R,5R,6R)-3-acetamido-4,5-diacetoxy-6-(acetoxymethyl) tetrahydro-2H-pyran-2-yl)oxy)pentanoic acid (11 g) was dissolved in dichloromethane (100 mL), then DCC (6.2 g) and pentafluorophenol (5.5 g) were added, and the reaction mixture was then stirred at room temperature until the reaction was completed. Upon completion, the reaction mixture was filtered and the filtrate was washed with water, dried and concentrated to give the crude product. After column separation and purification of the crude product (PE:EA=3:1-1:1), compound 7 was obtained;

(8) Compound 6 (2.46 g) and compound 7 (10 g) were dissolved in dichloromethane (50 mL), then DIEA (8 mL) was added to the reaction mixture, which was then stirred at room temperature until the reaction was completed. Upon reaction, dichloromethane and water was added to the reaction mixture for extraction, after which the organic phase was dried and concentrated to yield a crude product. After column separation and purification of the crude product (dichloromethane:methanol=10%-20%), compound 8 was obtained;

(9) Glutaric anhydride (10 g) and propargylamine (4.82 g) were dissolved in tetrahydrofuran (50 mL), and then the reaction was stirred at room temperature until it was completed. Upon completion, the reaction mixture was concentrated to dryness and compound 12 was obtained;

(10) Compound 12 (3.7 g) was dissolved in dichloromethane (50 mL), and then DCC (5.41 g), pentafluorophenol (4.83 g) were added to the reaction mixture, which was then stirred at room temperature until the reaction was completed. Upon completion, the reaction mixture was filtered and the filtrate was washed with water, dried and then concentrated to yield the crude product. After column separation and purification of the crude product (PE:EA=3:1-1:1), compound 13 was obtained;

(11) Compound 13 (6.5 g) and aminocaproic acid (2.54 g) were dissolved in tetrahydrofuran (50 mL), then the reaction mixture was stirred at room temperature until the reaction was completed. Upon completion, the reaction mixture was concentrated to dryness. Dichloromethane and water were added to the residue for extraction. The organic phase was dried and further concentrated to yield a crude product. After column separation and purification (dichloromethane:methanol=3%-8%) of the crude product, compound 14 was obtained;

(12) Compound 14 (15 g) was dissolved in dichloromethane (100 mL), and then DCC (3.94 g), pentafluorophenol (3.52 g) were added to the reaction mixture, which was then stirred at room temperature until the reaction was completed. Upon completion, the reaction mixture was filtered and the filtrate was washed with water, and then dried and concentrate to yield the crude product. After column separation and purification of the crude product (PE:EA=3:1-1:1), compound 15 was obtained;

(13) Compound 8 (0.34 g) and compound 15 (0.09 g) were dissolved in THF (5 mL), then anhydrous copper sulfate (0.092 g), and sodium ascorbate 0.146 g in an aqueous solution (5 mL) were added to the reaction mixture, which was then stirred at room temperature until the reaction was completed. Upon completion, THF was removed from the reaction mixture through concentration. The resulting residue was diluted with dichloromethane, decolored by activated carbon, and dried with anhydrous sodium sulfate. The resulting solution was filtered to remove the insoluble, and then concentrated to yield a crude product. After column separation and purification of the crude product (dichloromethane:methanol=10%-15%), GalNAc-1 was obtained.

Result

GalNAc-1 was obtained as a foam-like white solid (0.4 g). ¹H NMR (400 MHz, CDCl₃) δ:ppm 7.69 (s, 1H), 7.36 (dd, 4H), 6.97 (t, 3H), 6.81 (d, 1H), 6.74 (d, 2H), 6.40 (s, 1H), 5.35 (t, 3H), 5.19 (dd, 3H), 4.61 (d, 3H), 4.51 (d, 2H), 4.32 (s, 2H), 4.13 (m, 9H), 3.92 (dd, 6H), 3.65 (d, 6H), 3.50 (m, 3H), 3.27 (m, 18H), 2.68 (t, 2H), 2.44 (t, 6H), 2.33 (t, 2H), 2.21-1.26 (m, 61H). MS (ESI-TOF): m/z (M+H)⁺ 2283.44, (M+Na)⁺ 2305.41.

### Example 2. Preparation of GalNAc-2

Synthetic Scheme

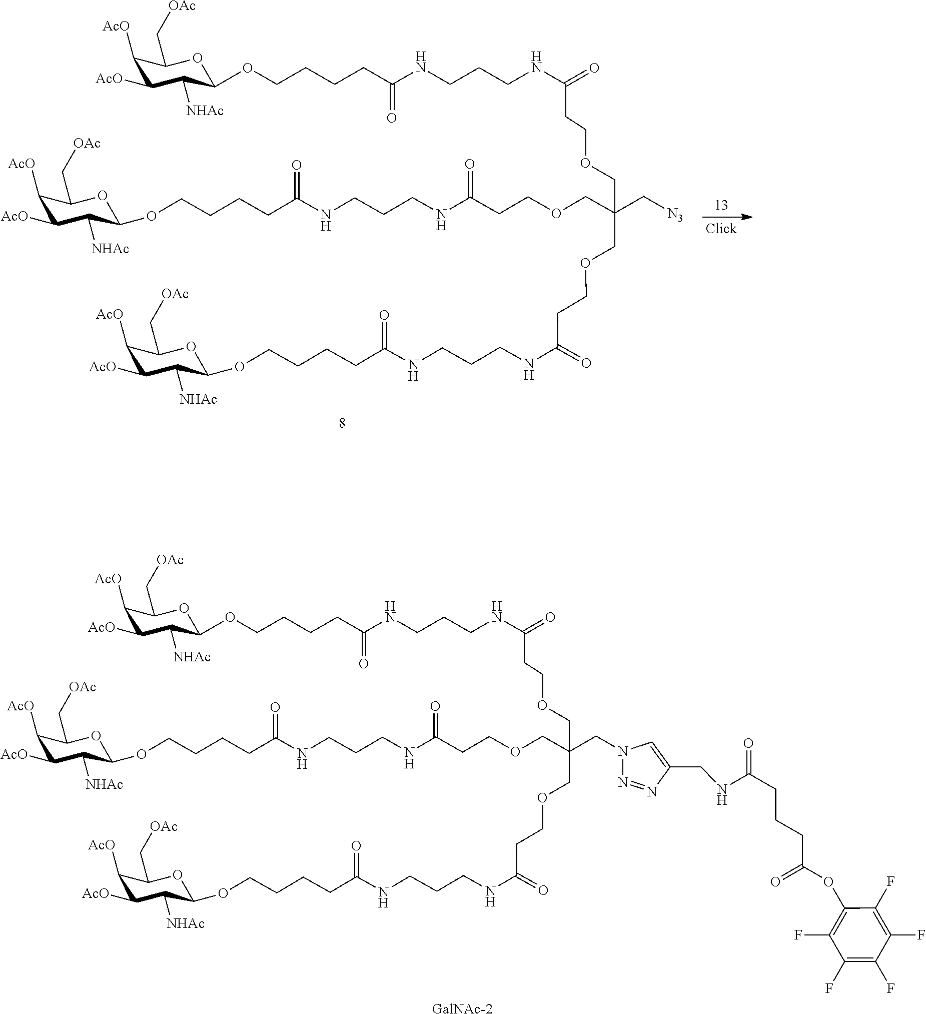

Compound 8 and 13 were synthesized as described in Example 1.

Procedure

GalNAc-2 was prepared using the method described in Example 1.

Result

GalNAc-2 was obtained as a white foam-like solid (0.2 g). ¹HNMR (400 MHz, CDCl₃) δ:ppm 7.73 (s, 1H), 6.90-6.40 (m, 10H), 5.36 (d, 3H), 5.08 (m, 3H), 4.60 (s, 3H), 4.50 (d, 2H), 4.35 (s, 2H), 4.14 (m, 9H), 3.92 (s, 6H), 3.66 (s, 6H), 3.51 (m, 3H), 3.27 (m, 18H), 2.79 (t, 2H), 2.45 (m, 6H), 2.25-1.80 (m, 44H), 1.80-1.26 (m, 20H MS(ESI-TOF): m/z (M+Na)⁺ 2191.38.

### Example 3. Preparation of GalNAc-3

Synthetic Scheme

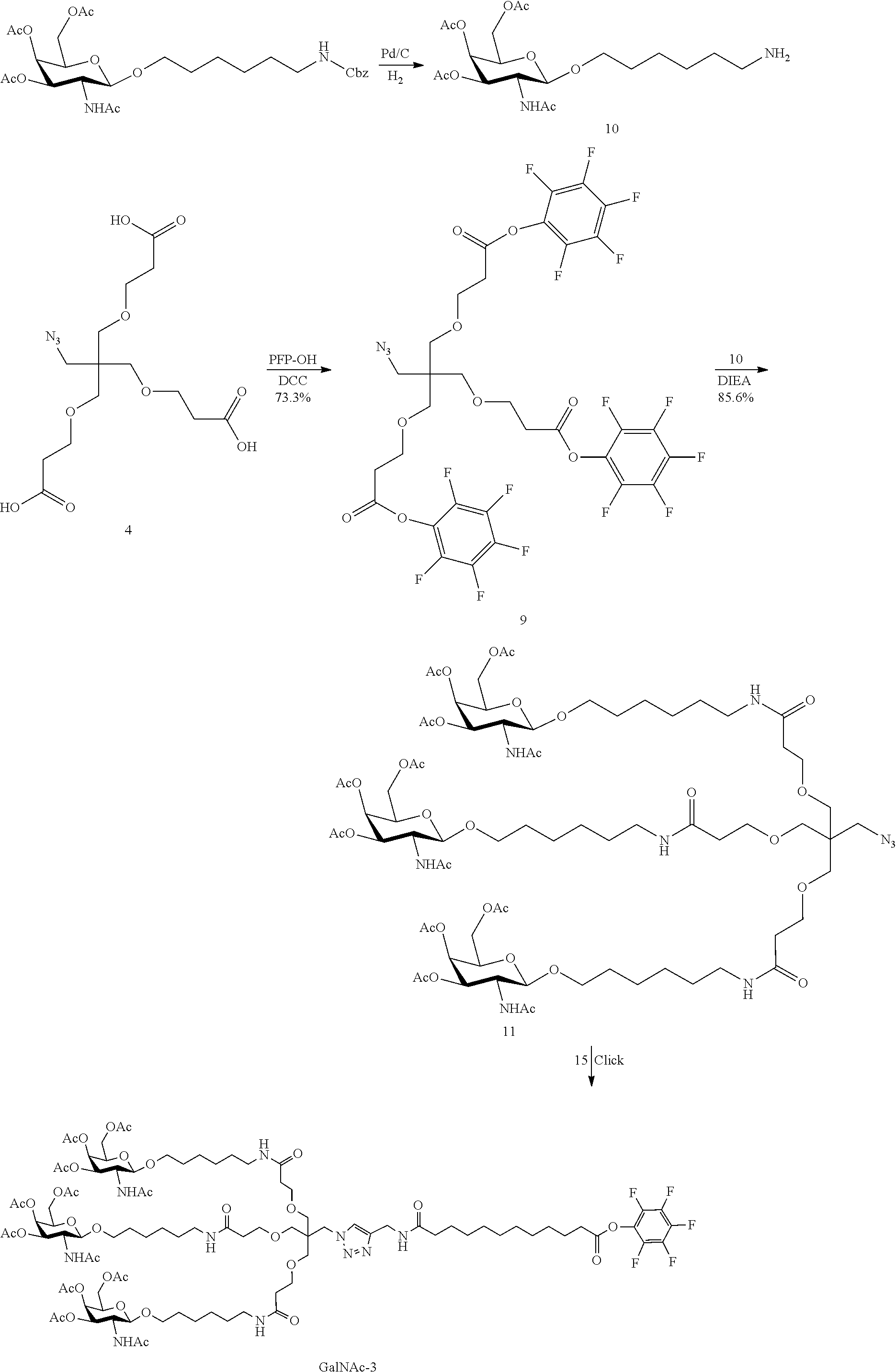

Compound 4 and 15 were the same as described in example 1.

Procedure

Compound 9, compound 11 and GalNAc-3 were prepared using the methods for preparation of compound 7, compound 8, and GalNAc-1 described in Example 1, respectively.

Cbz-hexylamine-galactose (5.5 g, purchased with Alading) was dissolved in ethyl acetate (100 mL), and then Pd/C (10%, 1 g) was added to the reaction mixture, which was further stirred in a hydrogen atmosphere at room temperature before the reaction was completed. Upon completion of the hydrogenation reaction, the reaction mixture was filtered and the filtrate was dried and concentrated to yield compound 10.

Result

GalNAc-3 was obtained as a white foam-like solid (0.2 g). ¹HNMR (400 MHz, CDCl₃) δ:ppm. 7.47-6.32 (m, 9H), 5.36 (s, 3H), 5.28 (m, 3H), 4.69 (s, 3H), 4.54 (s, 2H), 4.36 (m, 2H), 4.15 (m, 6H), 4.05 (m, 3H), 3.94 (dd, 6H), 3.66 (s, 6H), 3.47 (m, 3H), 3.25 (m, 12H), 2.68 (t, 2H), 2.44 (m, 6H), 2.30-1.80 (m, 42H), 1.80-1.26 (m, 32H).

MS(ESI-TOF): m/z (M+Na)⁺ 2133.43.

### Example 4. Preparation of GalNAc-4

Synthetic Scheme

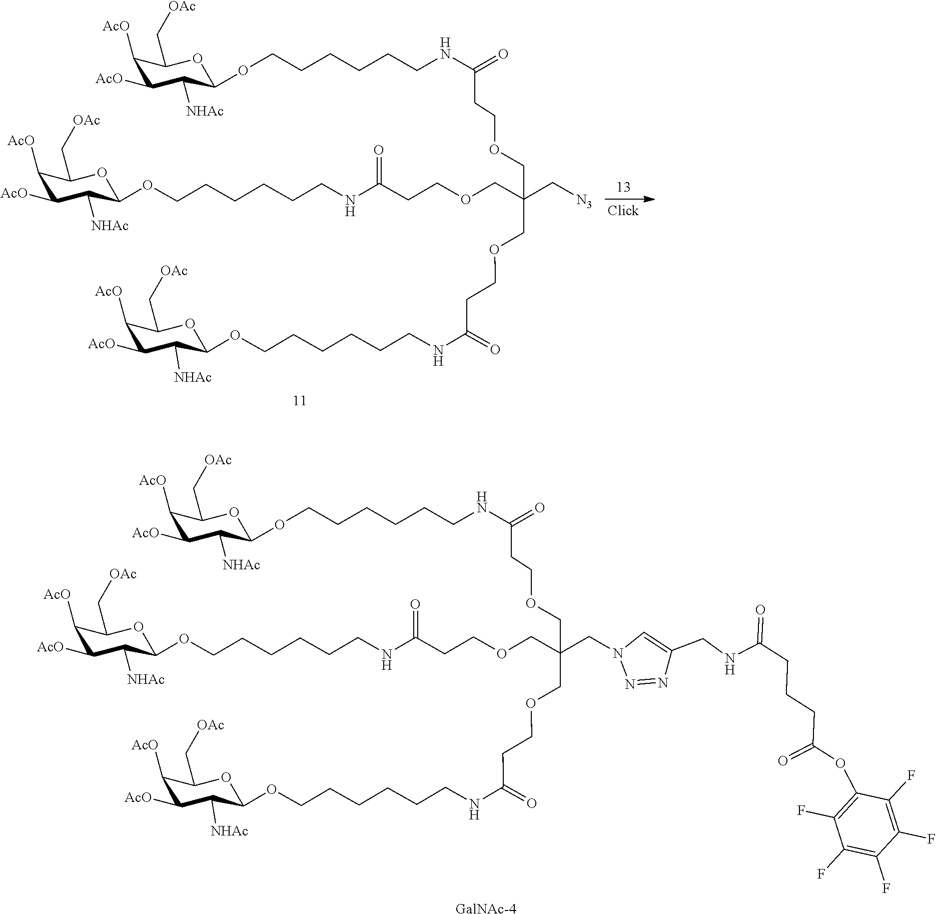

Compounds 11 and 13 were prepared using methods described in Examples 1 and 3, respectively.

Procedure

GalNAc-4 was prepared from reacting compound 11 and 13 using the method described in example 1.

Result

GalNAc-4 was obtained as a white foam-like solid (0.2 g). ¹HNMR (400 MHz, CDCl₃) δ:ppm. 7.74-6.43 (m, 8H), 5.36 (d, 3H), 5.28 (m, 3H), 4.68 (d, 3H), 4.53 (d, 2H), 4.31 (s, 2H), 4.14 (ddd, 6H), 4.05 (dd, 3H), 3.89 (ddd, 6H), 3.67 (m, 6H), 3.46 (dd, 3H), 3.24 (s, 12H), 2.78 (t, 2H), 2.43 (m, 6H), 2.30-1.80 (m, 38H), 1.80-1.26 (m, 26H).

MS(ESI-TOF): m/z (M+H)⁺ 1998.43, (M+Na)⁺ 2020.42.

### Example 5. Preparation of GalNAc-5

Synthetic Scheme

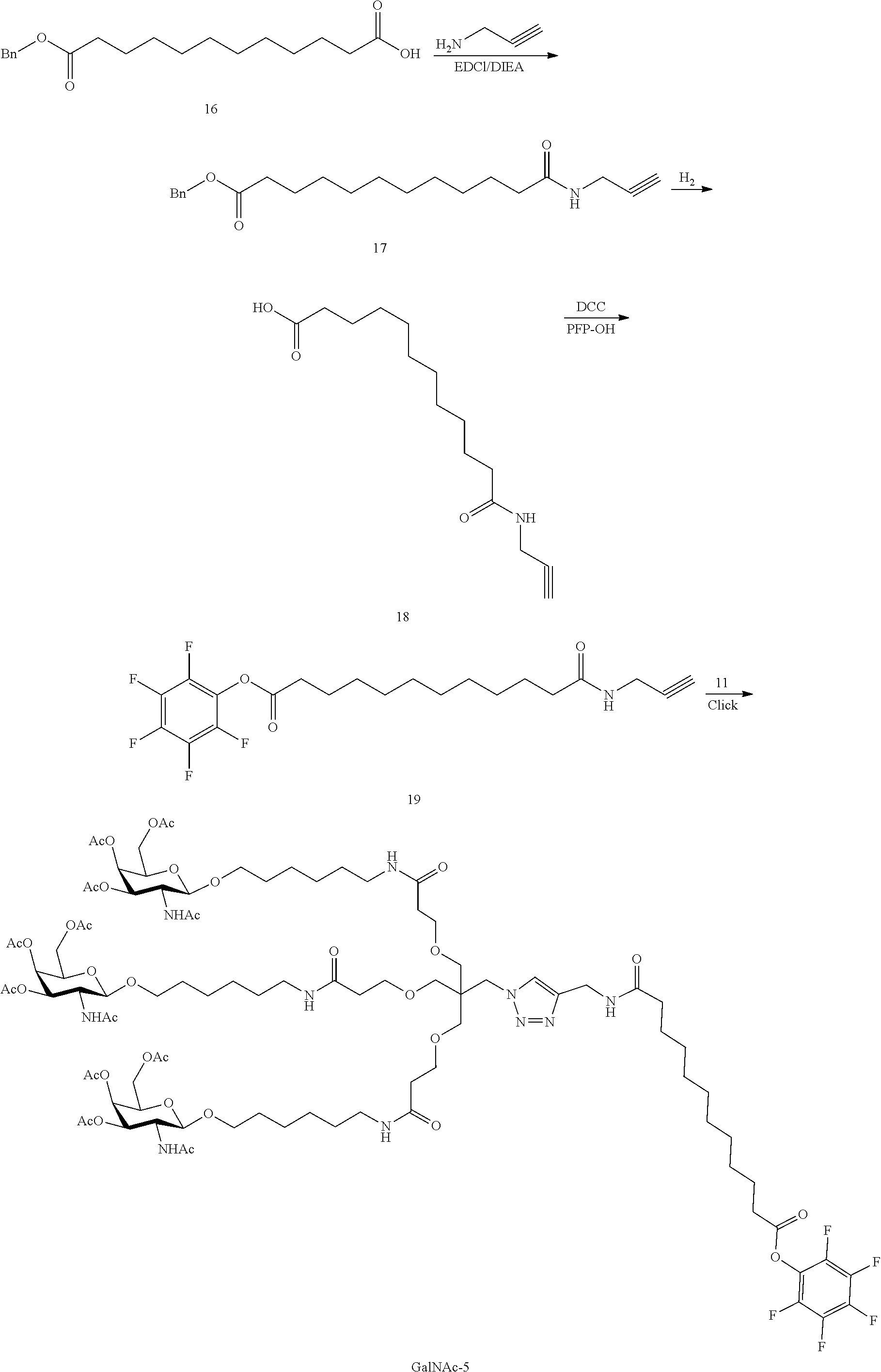

Compound 11 was the same as described in Example 3.

Procedure

(1) Compound 16 (1.6 g) was dissolved in dichloromethane (50 ml), then HOBt (2.23 g), EDCl (3.0 g), DIEA (3.3 g), and propargylamine (0.3 g) were added to the mixture. The reaction was then stirred at room temperature until the reaction was completed. Upon completion, dichloromethane and water were added to the reaction mixture for extraction, after which the organic phase was dried and concentrated to yield the crude product. After column separation and purification of the crude product (dichloromethane:methanol=2%-5%), compound 17 was obtained;

(2) Compound 17 (2 g) was added to methanol (10 mL), and then Pd/C (10%, 0.5 g) was added to the reaction mixture, which was further stirred in a hydrogen atmosphere at room temperature before the reaction was completed. Upon completion of the hydrogenation reaction, the reaction mixture was filtered and the filtrate was dried and concentrated to yield compound 18;

(3) PFP-laurate (4.5 g) was dissolved in dichloromethane (100 mL), then DCC (3.94 g) and pentafluorophenol (3.53 g) were added to the reaction mixture, which was then stirred at room temperature until the reaction was completed. Upon completion, the reaction mixture was filtered and the filtrate was washed with water, dried and concentrated to yield the crude product. After column separation and purification of the crude product (PE:EA=3:1-1:1), compound 19 was obtained;

(4) GalNAc-5 was prepared from reacting compound 11 and 19 using the method described in Example 1.

Result

GalNAc-5 was obtained as a white foam-like solid (0.4 g). ¹HNMR (400 MHz, CDCl₃) δ:ppm. 7.73-6.40 (m, 8H), 5.35 (d, 3H), 5.26 (m, 3H), 4.67 (d, 3H), 4.51 (s, 2H), 4.33 (s, 2H), 4.11 (dd, 6H), 4.07 (d, 3H), 3.89 (dt, 6H), 3.66 (m, 6H), 3.46 (dd, 3H), 3.24 (s, 12H), 2.75 (t, 2H), 2.41 (m, 6H), 2.25-1.80 (m, 38H), 1.80-1.26 (m, 40H). MS(ESI-TOF): m/z (M+H)⁺ 2096.19, (M+Na)⁺ 2119.20.

### Example 6. Preparation of GalNAc-6

Synthetic Scheme

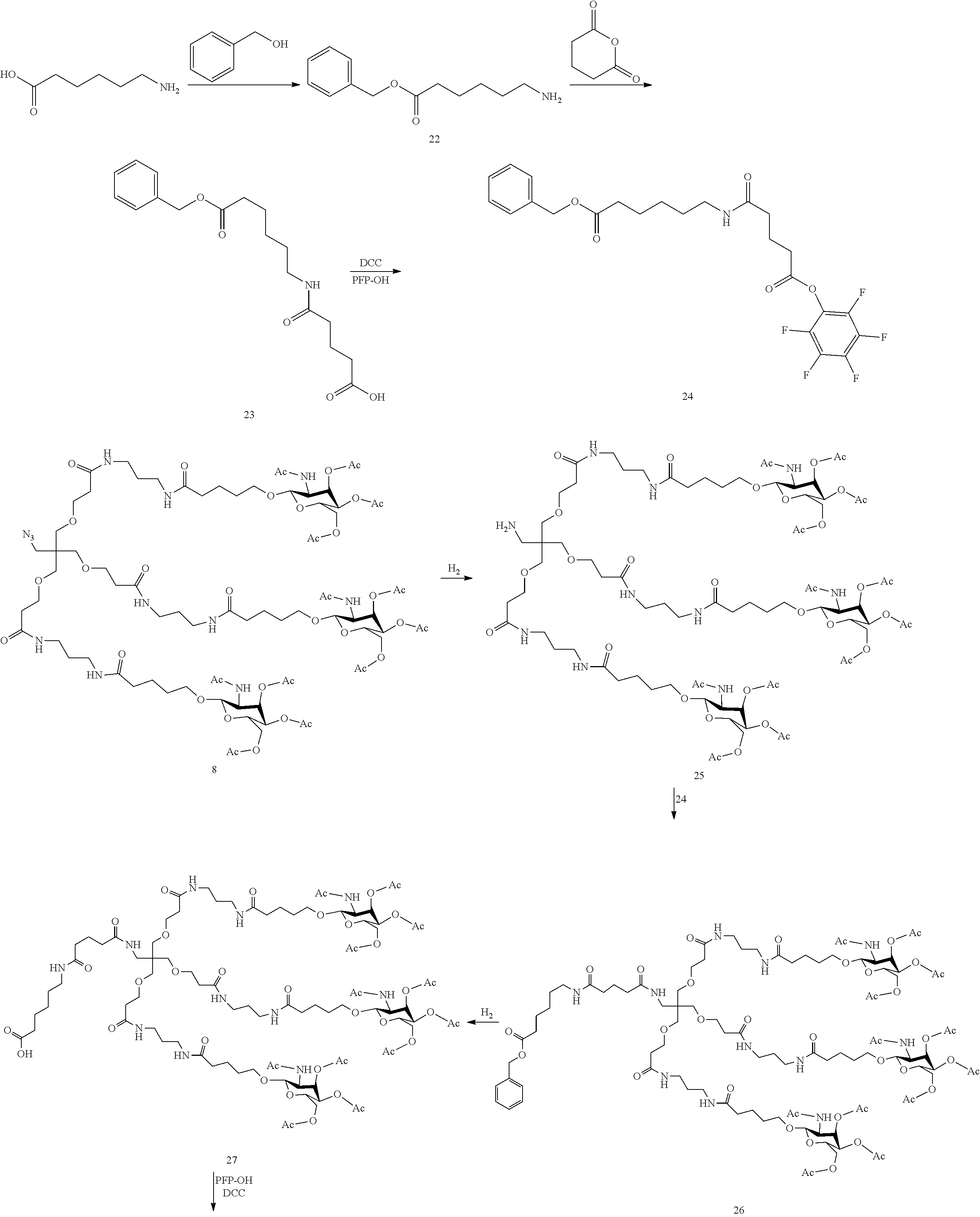

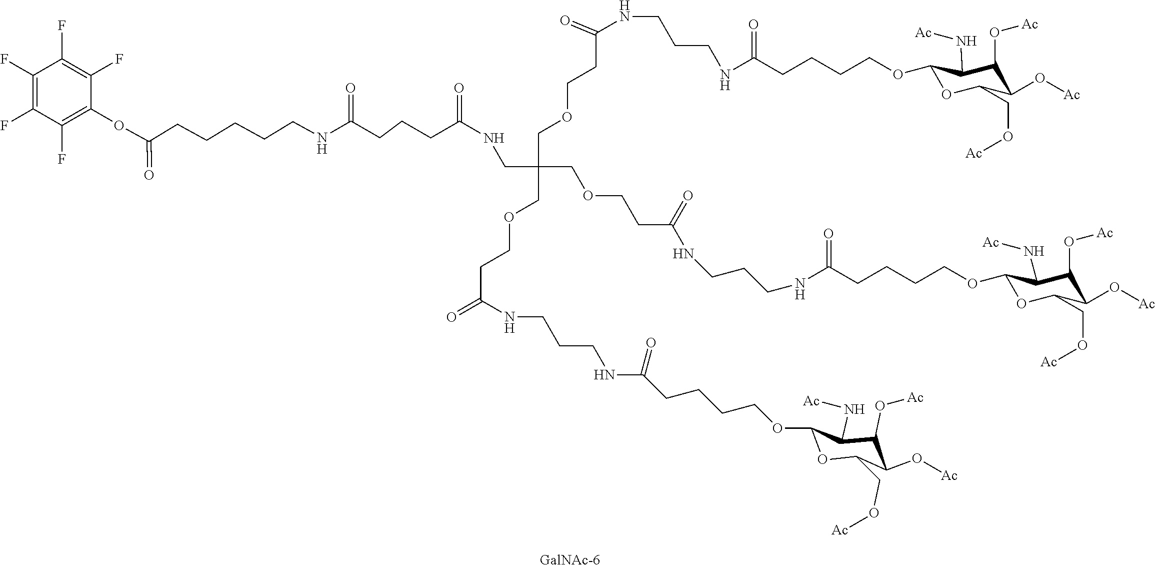

Compound 8 was prepared using the method described in Example 1.

Procedure

(1) Aminocaproic acid (47.9 g) was dissolved in toluene (700 mL), then benzyl alcohol (57 mL) and 4-toluenesulfonic acid (74 g) were added to the reaction mixture, which was then heated up to 115° C. while being stirred until the reaction was completed. Upon completion, the reaction mixture was cooled down to room temperature while being stirred, after which a large amount of solid precipitated. Methyl tert-butyl ether (500 mL) was added to the reaction mixture, which was then filtered. The solid was collected and dried to obtain compound 22; (2) Compound 22 (19 g) was dissolved in dichloromethane (100 mL), then glutaric anhydride (11 g) in dichloromethane (100 mL) was added to the reaction mixture, which was then stirred at room temperature until the reaction was completed. Upon completion, the reaction mixture was concentrated to yield compound 23; (3) Compound 23 (5 g) was dissolved in dichloromethane (100 mL), then DCC (3.6 g) and pentafluorophenol (3.3 g) were added to the reaction mixture, which was then stirred at room temperature before the reaction was completed. Upon completion, the reaction mixture was filtered and the filtrate was concentrated to yield the crude product. After column separation and purification of the crude product (PE:EA=3:1-2:1), compound 24 was obtained; (4) Compound 8 (0.13 g) was dissolved in ethyl acetate (10 mL) and anhydrous methanol (10 mL), then Pd/C (0.2 g, 10%) and 3 drops of acetic acid were added to the reaction mixture, which was then stirred in a hydrogen atmosphere at room temperature until the reaction was completed. Upon completion, the reaction mixture was filtered and the filtrate was concentrated to yield compound 25; (5) Compound 25 (2.8 g) was dissolved in dichloromethane (50 ml), then DIEA (0.5 g) and compound 24 (0.78 g) were added to the reaction mixture, which was then stirred at room temperature until the reaction completed. Upon reaction, the reaction mixture was concentrated to dryness. After column separation and purification of the crude product (dichloromethane:methanol 15%-25%), compound 26 was obtained; (6) Compound 26 (2.5 g) was dissolved in methanol (30 mL) and ethyl acetate (30 mL), then Pd/C (1.3 g, 10%) was added to the reaction mixture, which was then stirred in a hydrogen atmosphere at room temperature until the reaction was completed. Upon reaction completion, the reaction mixture was filtered and the filtrate was concentrated to yield compound 27; (7) Compound 27 (0.8 g) was dissolved in dichloromethane, then DCC (0.12 g), pentafluorophenol (0.11 g) were added to the reaction mixture, which was then stirred at room temperature until the reaction was completed. Upon completion, dichloromethane was added to the reaction mixture, which was then concentrated to yield the crude product. After column separation and purification of the crude product (dichloromethane:methanol=10%-15%), GalNAc-6 was obtained.

Result

GalNAc-6 was obtained as a white foam-like solid (0.7 g). ¹HNMR (400 MHz, d-DMSO) δ:ppm. 7.75-7.26 (m, 11H), 5.85 (d, 3H), 5.59 (d, 3H), 5.11 (dd, 3H), 4.63 (m, 6H), 4.50 (d, 6H), 4.37 (s, 3H), 4.17 (d, 6H), 4.07 (s, 3H), 3.90-3.70 (m, 18H), 3.28 (m, 2H), 2.92 (d, 6H), 2.80-2.25 (m, 52H), 2.25-1.26 (m, 24H). MS(ESI-TOF): m/z (M+H)⁺ 2201.82, (M+Na)⁺ 2223.86.

### Example 7. Preparation of GalNAc-7

Synthetic Scheme

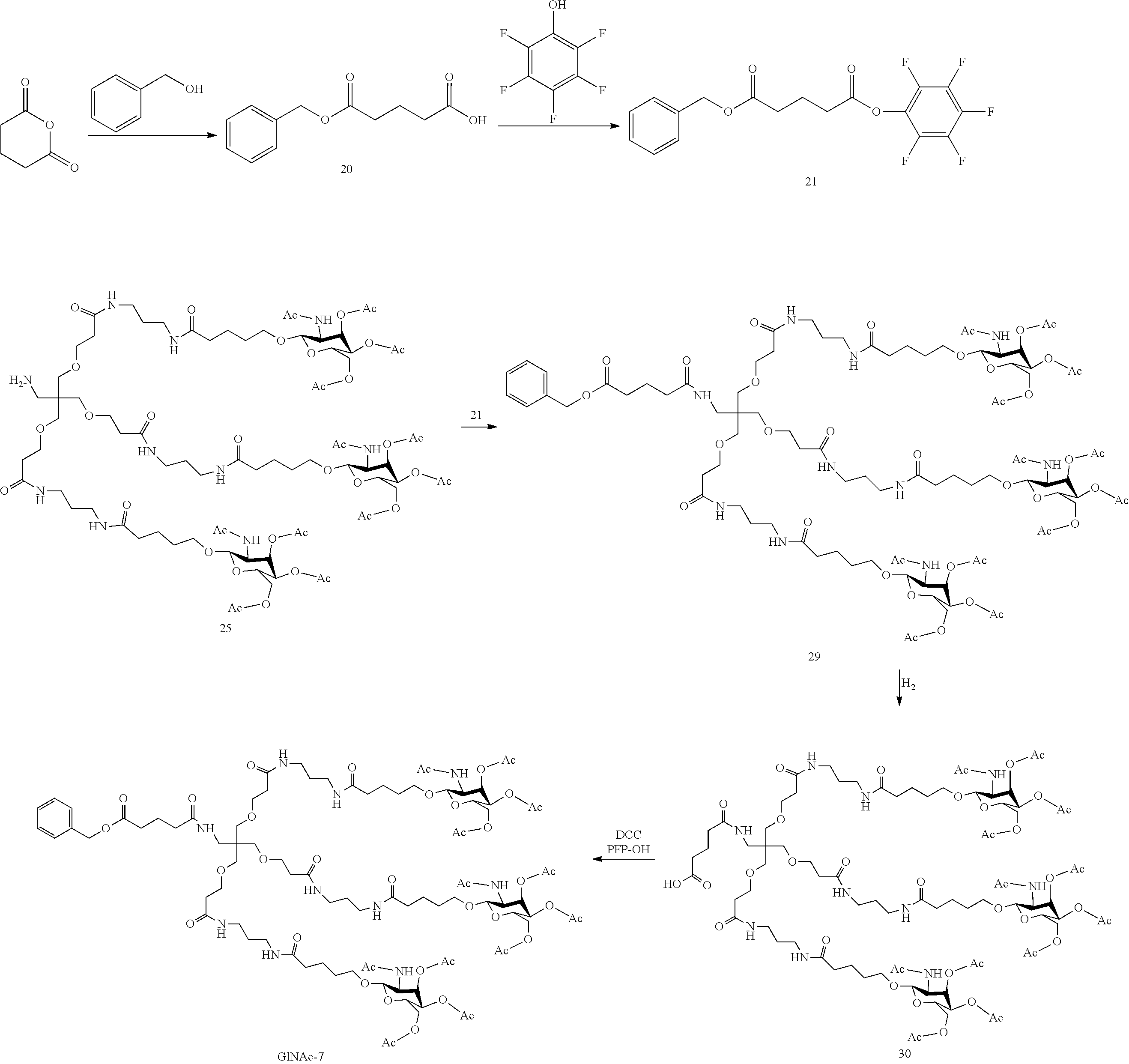

Compound 25 was prepared as described in Example 6.

Procedure

Compound 21 was prepared using the method for preparation of compound 13 described in Example 1.

Compounds 29, 30 and GalNAc-7 were prepared using the methods for preparation of compounds 26, 27 and GalNAc-6 in Example 6, respectively.

Result

GalNAc-7 was obtained as a white foam-like solid (0.5 g). ¹HNMR (400 MHz, CDCl₃) δ:ppm 7.32-6.63 (m, 10H), 5.35 (s, 3H), 5.20 (s, 3H), 4.62 (d, 3H), 4.13 (ddd, 9H), 3.92 (d, 6H), 3.66 (dd, 6H), 3.52 (d, 3H), 3.33 (m, 20H), 2.77 (t, 2H), 2.44 (s, 6H), 2.25-1.80 (m, 44H), 1.80-1.26 (m, 20H). MS(ESI-TOF): m/z (M+H)⁺ 2088.08, (M+Na)⁺ 2109.36.

### Example 8. Preparation of GalNAc-8

Synthetic Scheme

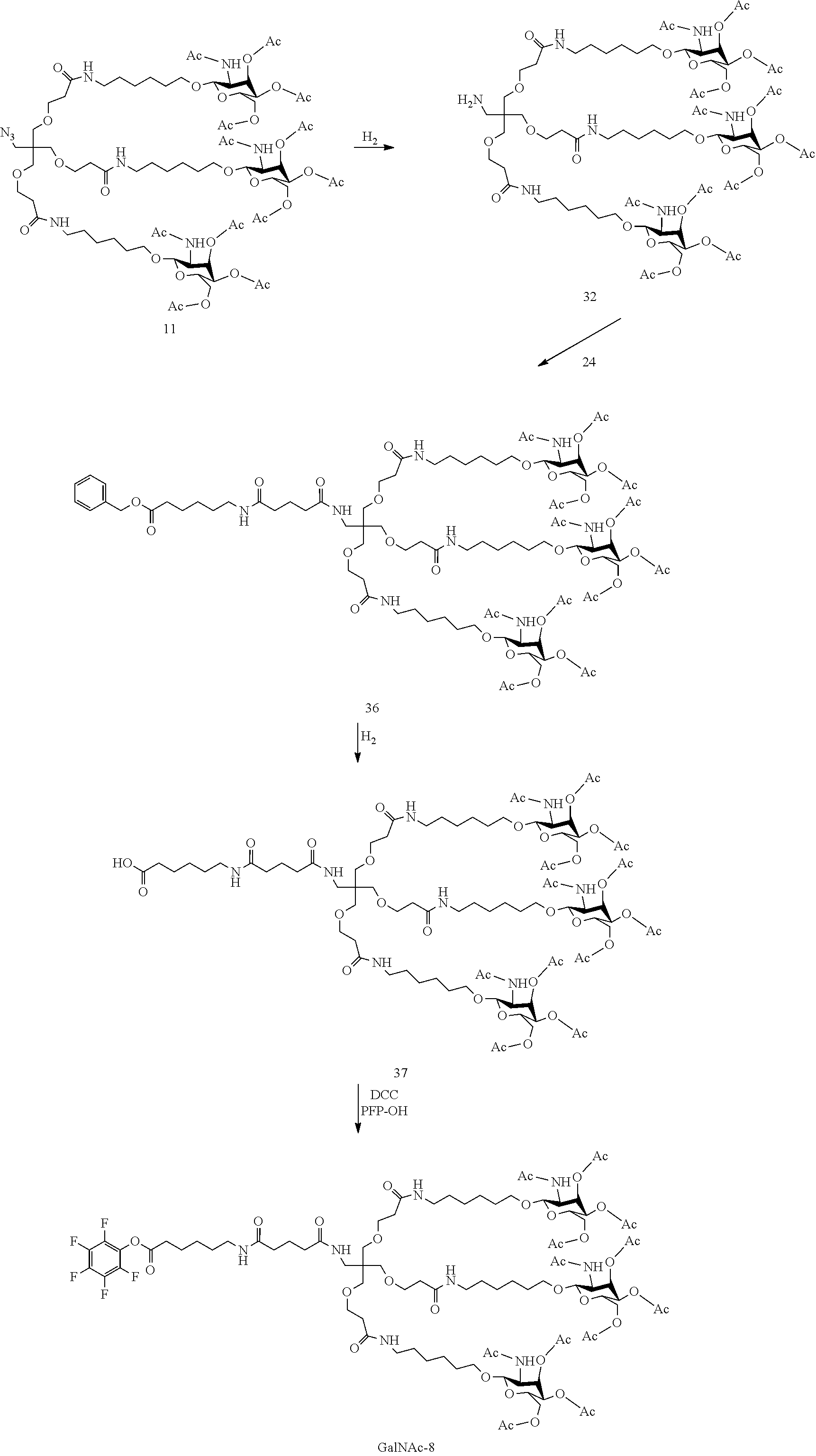

Compounds 11 and 24 were prepared as described in Examples 3 and example 6, respectively.

Procedure

Compounds 32, 36, 37, and GalNAc-8 were prepared using the methods for preparation of compounds 25, 26, 27 and GalNAc-6 in Example 6.

Result

¹HNMR (400 MHz, CDCl₃) δ:ppm. 7.7.06-6.37 (m, 8H), 5.36 (d, 3H), 5.270 (m, 3H), 4.69 (t, 3H), 4.15 (m, 6H), 4.00 (m, 3H), 3.90 (m, 6H), 3.65 (t, 6H), 3.47 (dt, 3H), 3.28 (m, 16H), 2.68 (t, 2H), 2.42 (m, 6H), 2.29 (t, 4H), 2.15-1.80 (s, 36H), 1.80-1.26 (m, 32H).

MS(ESI-TOF): m/z (M+H)⁺ 2030.38, (M+Na)⁺ 2052.41.

### Example 9. Preparation of GalNAc-9

Synthetic Scheme

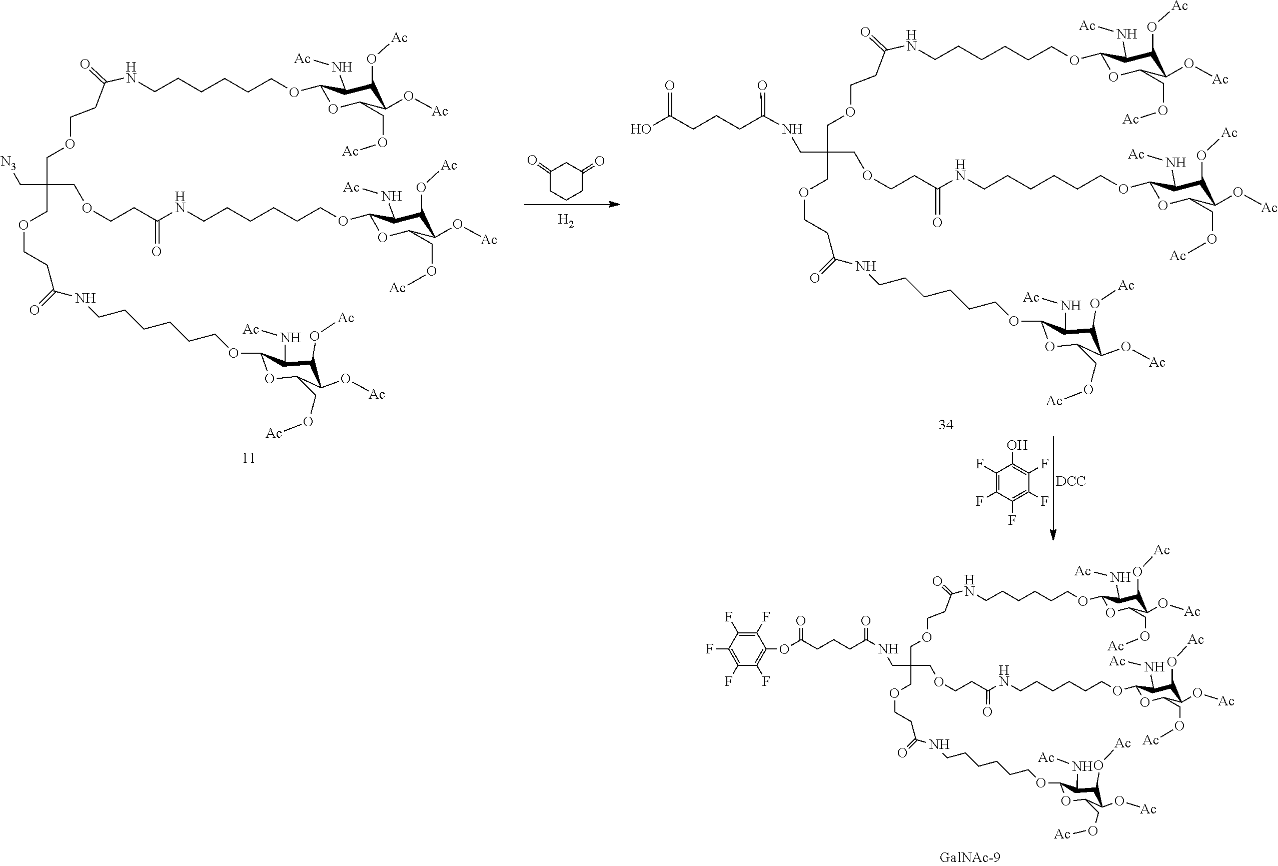

Compound 11 was prepared as described in Example 3.

Procedure

(1) Azide compound 11 (1.3 g) was dissolved in THF (15 mL), then Pd/C (0.3 g) and glutaric anhydride (0.11 g) were added to the reaction mixture, which was then stirred at room temperature in a hydrogen atmosphere until the hydrogenation reaction was completed. Upon completion, the reaction mixture was filtered and the filtrate was collected and concentrated the reaction residue. Dichloromethane and water were added to the reaction residue for extraction. The extracted organic phase was concentrated and concentrated to yield the crude product. After column separation and purification of the crude product (dichloromethane:methanol=10%-15%), compound 34 (0.8 g) was obtained; (2) GalNAc-9 was prepared using the method for preparation of GalNAc-6 in example 6.

Result

GalNAc-9 was obtained as a white foam-like solid (0.8 g).

¹HNMR (400 MHz, d-DMSO) δ:ppm. 7.63 (s, 1H), 7.42 (s, 3H), 7.17 (s, 3H), 5.85 (d, 3H), 5.60 (t, 3H), 5.10 (dd, 3H), 4.63 (td, 6H), 4.49 (m, 6H), 4.34 (d, 3H), 4.16 (m, 6H), 4.03 (d, 3H), 3.84 (d, 6H), 3.73 (d, 6H), 3.35 (m, 2H), 2.91 (d, 6H), 2.70-2.40 (m, 42H), 2.21-1.76 (m, 24H).

MS(ESI-TOF): m/z (M+H)⁺ 1917.93, (M+Na)⁺ 1938.69.

### Example 10. Preparation of GalNAc-10

Synthetic Scheme

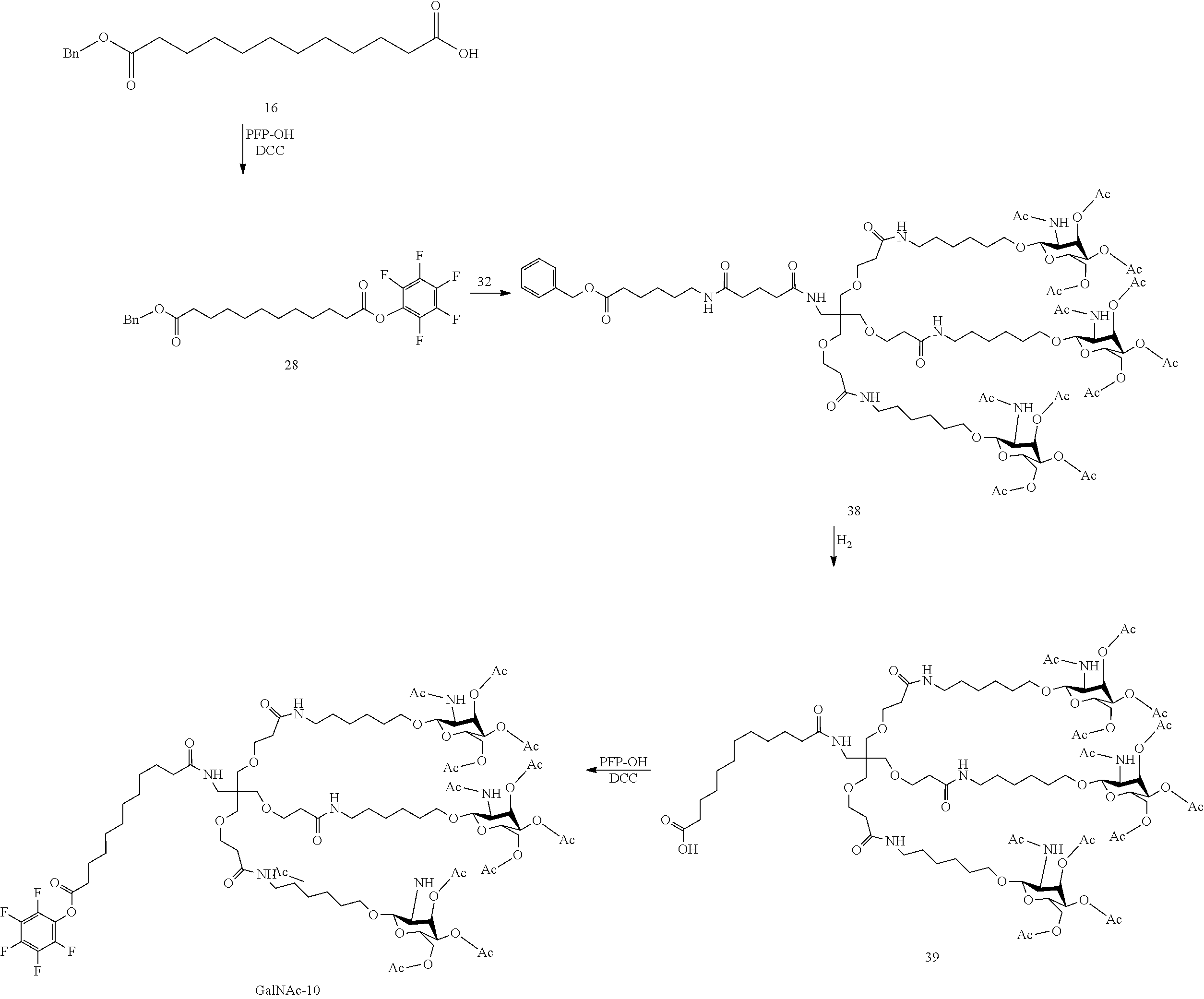

Compound 28 in the present example was prepared as described for the synthesis of compound 24 in Example 6.

Procedure

(1) Amino compound 32 (3 g) was dissolved in dichloromethane (30 mL), then DIEA (1 ml) and compound 28 (1.31 g) were added to the reaction mixture, which was then stirred at room temperature until the reaction was completed. Upon completion, dichloromethane was added to the reaction mixture and the resulting solution was concentrated to yield a crude product. After column separation and purification of the crude product (dichloromethane:methanol=10%-15%), compound 38 was obtained; (2) Compound 39 and GalNAc-10 were prepared using the methods for preparation of compound 27 and GalNAc-6 in example 6, respectively.

Result

GalNAc-10 was obtained as a white foam-like solid (0.45 g). ¹HNMR (400 MHz, CDCl₃) δ:ppm 7.7.06-6.40 (m, 7H), 5.35 (d, 3H), 5.28 (m, 3H), 4.67 (t, 3H), 4.14 (m, 6H), 4.02 (m, 3H), 3.93 (m, 6H), 3.61 (t, 6H), 3.48 (dt, 3H), 3.29 (m, 14H), 2.67 (t, 2H), 2.43 (m, 6H), 2.28 (t, 2H), 2.15-1.80 (s, 36H), 1.80-1.26 (m, 40H).

MS(ESI-TOF): m/z (M+H)⁺ 2015.11, (M+Na)⁺ 2038.47.

### Example 11. Preparation of GalNAc-11

Synthetic Scheme

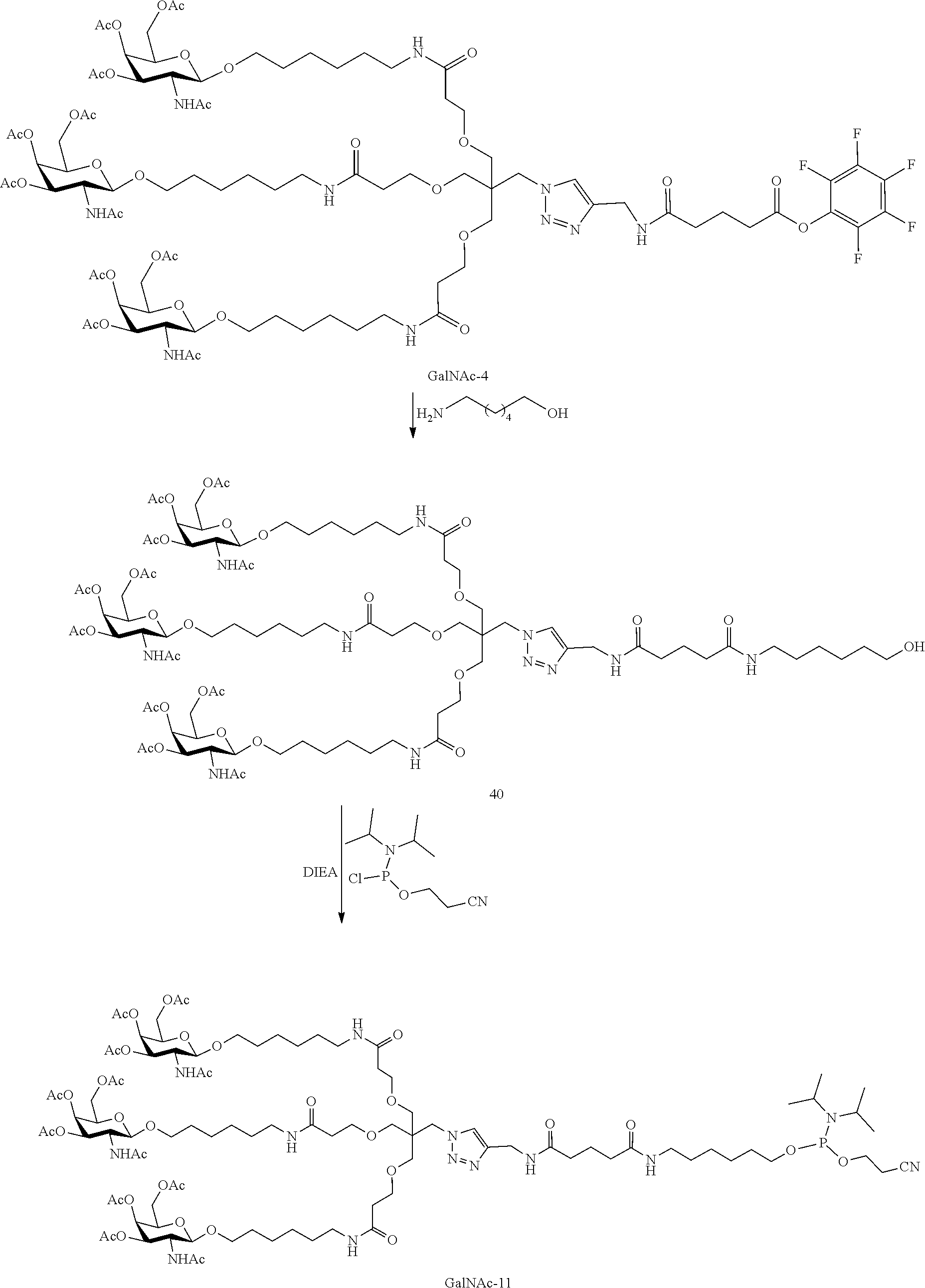

Compound GalNAc 4 is the same as described in example 4.

Procedure

(1) GalNAc-4 (3 g) was dissolved in dichloromethane (30 mL), then DIEA (0.3 ml), 1-hydroxyhexanoic acid (0.18 g) were added to the reaction mixture, which was then stirred at room temperature until the reaction was completed. Upon completion, dichloromethane was added to the reaction mixture, which was then concentrated to yield the crude product. After column separation and purification of the crude product (dichloromethane:methanol 10%-15%), compound 40 was obtained; (2) Compound 40 (1 g) was dissolved in anhydrous dichloromethane (10 mL), then the reaction mixture was cooled to 0° C. before DIEA (0.2 ml), and phosphorous oxychloride (0.14 g) was added. Then the reaction was stirred at 0° C. until it was completed. Upon completion, dichloromethane was added to the reaction mixture, which was then dried and concentrated to yield the crude product. After column separation and purification of the crude product (dichloromethane:methanol 10%-15%), GalNAc-11 was obtained.

Result

Compound 40: 1HNMR (400 MHz, CDCl3) δ:ppm. 7.71-6.38 (m, 9H), 5.36 (d, 3H), 5.27 (m, 3H), 4.68 (d, 3H), 4.53 (s, 2H), 4.31 (s, 2H), 4.15 (m, 6H), 4.01 (dd, 3H)), 3.89 (ddd, 6H), 3.65 (dd, 8H), 3.46 (m, 3H), 3.24 (m, 14H), 2.46 (d, 6H), 2.31 (d, 2H), 2.23 (s, 2H), 2.16-1.80 (m, 36H), 1.80-1.26 (m, 34H).

MS(ESI-TOF): m/z (M−H)⁻ 1929.81.

GalNAc-11 was obtained as a white foam-like solid (0.5 g). MS(ESI-TOF): m/z (M+Na)⁺ 2153.62.

### Example 12. Preparation of GalNAc-12

Synthetic Scheme

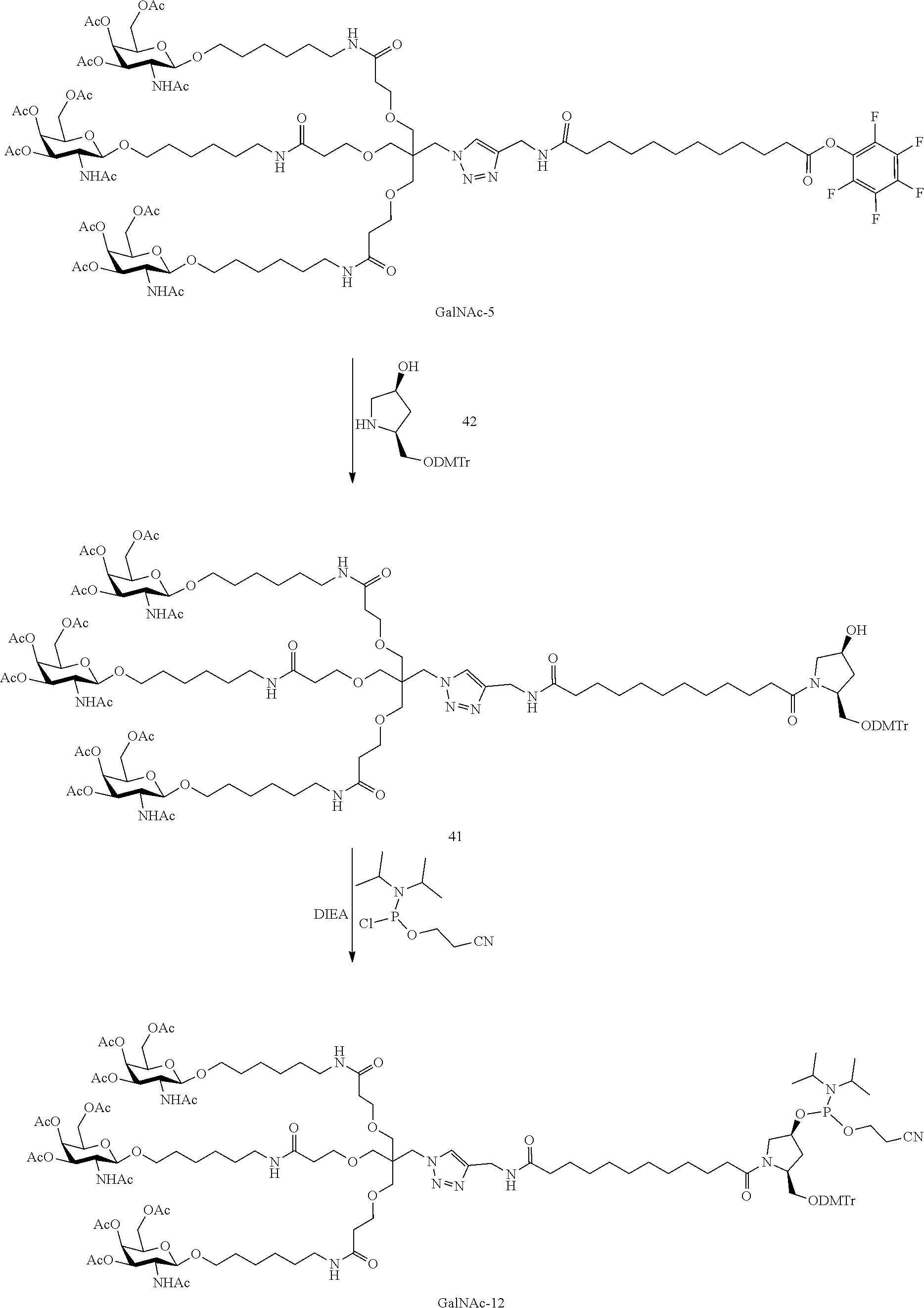

Compound 42 is purchased from Alading; compound GalNAc-5 is the same as described in Example 5.

Procedure

(1) GalNAc-5 (e.g., 2 g) is dissolved in dichloromethane (ca. 20 mL), then DIEA (ca. 0.3 ml), compound 42 (ca. 0.44 g) are added to the reaction, which is then stirred at room temperature before the reaction is deemed complete. Upon completion, dichloromethane is added to the reaction mixture, which is then dried and concentrated to yield the crude product. After column separation and purification of the crude product (dichloromethane:methanol using the appropriate gradient, e.g., 10%-15% methanol), compound 41 is obtained; (2) Compound 41 (e.g., 1 g) is dissolved in dry dichloromethane (ca. 10 mL), then the reaction mixture is cooled to 0° C. before DIEA (ca. 0.2 ml) and phosphorous oxychloride (ca. 0.12 g) are added. The reaction is then stirred at 0° C. until it is completed. Upon completion, dichloromethane is added to the reaction mixture, which is then dried and concentrated to yield the crude product. After column separation and purification of the crude product (dichloromethane:methanol under appropriate gradient, e.g., 10%-15% methanol), GalNAc-12 is obtained.

### Example 13. Preparation of Modified Single-Stranded Oligonucleotide (Antisense Strand)

The sequence discussed in Examples 13, and 16-18 are as follows: 5′-Cy5-mUmGmAfCmAmAmAfCmGmGmGfCmAmAftmAfUmAfC-3′ (SEQ ID NO: 1). In this example, the modified oligonucleotide was synthesized on a 1 μmol scale. The experiment was carried out as described below:

(1) 1 μmol of standard Controlled Pore Glass (CPG) solid-phase support or 3′-cholesterol-modified CPG solid-phase support (purchased from Chemgenes), or 3′-amino-modified CPG solid-phase support (purchased from Kinovite)] was weighed. 2′-O-TBDMS-protected RNA phosphoramidite monomers, DNA monomers, 2′-methoxy monomers, 2′-fluoromonomers (purchased from Sigma Aldrich); together with amino-C16-12-phosphoramidite (or fluorescent phosphoramidite) for synthesis of 5′ modification were dissolved in anhydrous acetonitrile to prepare a solution with a concentration of 0.2 M. For oligonucleotides with thio-modification on the phosphate backbone, 0.2 M PADS solution was used as a thio-reagent. An acetonitrile solution with 0.25 M of 5-ethylthio-1H-tetrazole (purchased from Chemgenes) as the activator, a pyridine/water solution with 0.02 M of iodine as the oxidant, and a chloromethane solution with 3% of trichloroacetic acid as the deprotection reagent were prepared and placed in the designated positions of a DNA/RNA automatic synthesizer (GE AKTA™ OP100).

(2) The desired oligonucleotide base sequence was entered in the synthesis program, then the oligonucleotide synthesis is started and repeated. Each step of coupling takes 6 minutes, and each step of galactose ligand monomer coupling takes 10-20 minutes. The solid-phase oligonucleotide synthesis is completed after the automatic circulation.

(3) The CPG was dried with dry nitrogen, and then transferred to a 5 mL EP tube. Ammonia/ethanol solution(3/1) (2 mL) was added to the EP tube, which was then heated at 55° C. for 16-18 hours. The resulting solution was centrifuged at 10,000 rpm for 10 min.

The supernatant layer was collected, and its ammonia solution/ethanol was removed with reduced pressure to yield a white gel-like solid. The solid was dissolved in a TBAF solution (1M in THF, 200 μL) and shaken for 20 hours at room temperature. To the resulting mixture was added Tris-HCl buffer (ph 7.4, 1M, 0.5 mL). The mixture thus obtained as shaken at room temperature for 15 minutes and centrifuged to % of the original volume. The resulting mixture was extracted twice with choloroform (0.5 mL) and treated with a TEAA sampling solution (I mL 0.1M) to provide a solution, which was then poured into a solid phase extraction column to remove excess salt in the solution.

(4) The concentration of the obtained oligonucleotide was determined by a micro-volume UV-visible spectrophotometer (KO5500). The mass spectrometry of the oligonucleotide was detected and analyzed on an Oligo HTCS LC-MS system (Novatia). After the first-level scan, the molecular weight was calculated through a normalized method on the Promass software.

### Example 14. Preparation of Modified Single-Stranded Oligonucleotide (Sense Strand)

The sequence discussed in Examples 14-18 are as follows: 5′-mGtUmAfUmGfUfUmGfCfCfCmGfUtUfUmGfUtCmA-3′ (SEQ ID NO: 2). In this example, the modified oligonucleotide was synthesized on 1 μmol scale. The experiment was carried out as described below.

(1) 1 μmol of standard Controlled Pore Glass (CPG) solid-phase support or 3′-cholesterol-modified CPG solid-phase support (purchased from Chemgenes), or 3′-amino-modified CPG solid-phase support (purchased from Kinovite)], DNA monomers, 2′-methoxy monomers, 2′-fluoromonomers (purchased from Sigma Aldrich), amino-C16-12-phosphoramidite (or fluorescent phosphoramidite) for synthesis of 5′-modification were dissolved in anhydrous acetonitrile to make a solution of 0.2 M. For oligonucleotides with thio-modification on the phosphate backbone, 0.2 M PADS solution was used as a thio-reagent. An acetonitrile solution (0.25 M) of 5-ethylthio-1H-tetrazole (purchased from Chemgenes) as the activator, a pyridine/water solution (0.02 M) of iodine as the oxidant, and a chloromethane solution (3%) of trichloroacetic acid as the deprotection reagent were prepared and placed in the designated position in the DNA/RNA automatic synthesizer (GE AKTAOP100).

(2) The desired oligonucleotide base sequence was entered in the synthesis program, then the oligonucleotide synthesis is started and repeated. Each step of coupling takes 6 minutes, and each step of galactose ligand monomer coupling takes 6-10 minutes. The solid-phase oligonucleotide synthesis is completed after the automatic circulation.

(3) The CPG was dried with dry nitrogen, and then transferred to a 5 mL EP tube. Ammonia solution (2 mL) was added to the EP tube, which was then heated at 55° C. for 16-18 hours. The resulting solution was centrifuged at 10,000 rpm for 10 min. The supernatant layer was collected, and its ammonia solution/ethanol was removed with reduced pressure to yield a white or yellow gel-like solid. TEAA sampling solution (1 mL 0.1M) was added to the solid to create a solution, which was then poured into a solid phase extraction column to remove excess salt in the solution.

(4) The concentration of the obtained oligonucleotide was determined by a micro-volume UV-visible spectrophotometer (KO5500). The mass spectrometry of the oligonucleotide was detected and analyzed on an Oligo HTCS LC-MS system (Novatia). After the first-level scan, the molecular weight was calculated through a normalized method on the Promass software.

### Example 15. Preparation of GalNAc-Modified Oligonucleotides

The amino-modified oligonucleotide (sense strand) prepared in Example 14 was dissolved in a buffer solution. Various GalNAc-pentafluorophenol esters (GalNAc selected from GalNAc-1 to GalNAc-10) dissolved in acetonitrile were respectively added to the amino-modified oligonucleotide solution, mixed well, and then reacted at room temperature for at least 3 hours. After the reaction is completed, the acetyl protecting group was removed, and the reaction product was purified by ion exchange chromatography (WATERS) using a linear gradient DNAPAc PA-00 ion exchange column. The mobile phase A solution was 20 mM NaOH, and the mobile phase B solution was 20 mM NaOH+2 M NaCl mixture.

TABLE 1
GalNAc-modified oligonucleotides (sense strand)

| | | Measured |
|---|---|---|
| Sample | | Molecular |
| Number | Structure (5′-3′) | Weight |
| Sense | (SEQ ID NO: 3)5′-GalNAc-1- | 7497.93 |
| strand- | mGfUmAfUmGfUfUmGfCfCfCmGf | |
| 101 | UfUfUmGfUfCmA3′ | |
| Sense | (SEQ ID NO: 4)5′-GalNAc-2- | 7669.08 |
| strand- | mGfUmAfUmGfUfUmGfCfCfCmGfU | |
| 102 | fUfUmGfUfCmA3′ | |
| Sense | (SEQ ID NO: 5)5′-GalNAc-3- | 7611.08 |
| strand- | mGfUmAfUmGfUfUmGfCfCfCmGfU | |
| 103 | fUfUmGfUfCmA3′ | |
| Sense | (SEQ ID NO: 6)5′-GalNAc-4- | 7782.24 |
| strand- | mGfUmAfUmGfUfUmGfCfCfCmGfU | |
| 104 | fUfUmGfUfCmA3′ | |
| Sense | (SEQ ID NO: 7)5′-GalNAc-5- | 7750.16 |
| strand- | mGfUmAfUmGfUfUmGfCfCfCmGfU | |
| 105 | fUfUmGfUfCmA3′ | |
| Sense | (SEQ ID NO: 8)5′-GalNAc-6- | 7579.00 |
| strand- | mGfUmAfUmGfUfUmGfCfCfCmGfU | |
| 106 | fUfUmGfUfCmA3′ | |
| Sense | (SEQ ID NO: 9)5′-GalNAc-7- | 7692.16 |
| strand- | mGfUmAfUmGfUfUmGfCfCfCmGfU | |
| 107 | fUfUmGfUfCmA3′ | |
| Sense | (SEQ ID NO: 10)5′-GalNAc-8- | 7863.31 |
| strand- | mGfUmAfUmGfUfUmGfCfCfCmGfUf | |
| 108 | UfUmGfUfCmA3′ | |
| Sense | (SEQ ID NO: 11)5′-GalNAc-9- | 7753.27 |
| strand- | mGfUmAfUmGfUfUmGfCfCfCmGfUf | |
| 109 | UfUmGfUfCmA3′ | |
| Sense | (SEQ ID NO: 12)5′-GalNAc-10- | 7810.32 |
| strand- | mGfUmAfUmGfUfUmGfCfCfCmGfUfU | |
| 110 | fUmGfUfCmA3′ | |

Note:
N = RNA; dN = DNA; mN = 2′OMe modification; and fN = 2′F modification.

Sequence of exemplary modified oligonucleotides and corresponding molecular weight (MW) detection results are shown in Table 1.

### Example 16. Preparation of Double-Stranded Oligonucleotides

The experiment was carried out as follows: The 5′-Cy5-antisense strand oligonucleotides synthesized in Example 13 were respectively mixed with the sense strand-GalNAc-1-10 oligonucleotides prepared in Example 15, according to the UV absorption ratio of 1:1. The mixture was incubated at 95° C. for 3 minutes in a heated water bath, then cooled to room temperature to form double-stranded oligonucleotides.

TABLE 2
Double-stranded RNA structures

| Sample | |
|---|---|
| number | Double-stranded structure (5′-3′) |
| 101 | (SEQ ID NO: 13)5′-GalNAc-1- |
| | mGfUmAfUmGfUfUmGfCfCfCmGfUfUfUmGfUfCmA-3′ |
| | (SEQ ID NO: 14)3′- |
| | fCmAfUmAfCmAmAfCmGmGmGfCmAmAmAfCmAmGmU- |
| | Cy5-5′ |
| 102 | (SEQ ID NO: 15)5′-GalNAc-2- |
| | mGfUmAfUmGfUfUmGfCfCfCmGfUfUfUmGfUfCmA3′ |
| | (SEQ ID NO: 16)3′- |
| | fCmAfUmAfCmAmAfCmGmGmGfCmAmAmAfCmAmGmU- |
| | Cy5-5′ |
| 103 | (SEQ ID NO: 17)5′-GalNAc-3- |
| | mGfUmAfUmGfUfUmGfCfCfCmGfUfUfUmGfUfCmA3′ |
| | (SEQ ID NO: 18)3′- |
| | fCmAfUmAfCmAmAfCmGmGmGfCmAmAmAfCmAmGmU- |
| | Cy5-5′ |
| 104 | (SEQ ID NO: 19)5′-GalNAc-4- |
| | mGfUmAfUmGfUfUmGfCfCfCmGfUfUfUmGfUfCmA3′ |
| | (SEQ ID NO: 20)3′- |
| | fCmAfUmAfCmAmAfCmGmGmGfCmAmAmAfCmAmGmU- |
| | Cy5-5′ |
| 105 | (SEQ ID NO: 21)5′-GalNAc-5- |
| | mGfUmAfUmGfUfUmGfCfCfCmGfUfUfUmGfUfCmA3′ |
| | (SEQ ID NO: 22)3′- |
| | fCmAfUmAfCmAmAfCmGmGmGfCmAmAmAfCmAmGmU- |
| | Cy5-5′ |
| 106 | (SEQ ID NO: 23)5′-GalNAc-6- |
| | mGfUmAfUmGfUfUmGfCfCfCmGfUfUfUmGfUfCmA3′ |
| | (SEQ ID NO: 24)3′- |
| | fCmAfUmAfCmAmAfCmGmGmGfCmAmAmAfCmAmGmU- |
| | Cy5-5′ |
| 107 | (SEQ ID NO: 25)5′-GalNAc-7- |
| | mGfUmAfUmGfUfUmGfCfCfCmGfUfUfUmGfUfCmA3′ |
| | (SEQ ID NO: 26)3′- |
| | fCmAfUmAfCmAmAfCmGmGmGfCmAmAmAfCmAmGmU- |
| | Cy5-5′ |
| 108 | (SEQ ID NO: 27)5′-GalNAc-8- |
| | mGfUmAfUmGfUfUmGfCfCfCmGfUfUfUmGfUfCmA3′ |
| | (SEQ ID NO: 28)3′- |
| | fCmAfUmAfCmAmAfCmGmGmGfCmAmAmAfCmAmGmU- |
| | Cy5-5′ |
| 109 | (SEQ ID NO: 29)5′-GalNAc-9- |
| | mGfUmAfUmGfUfUmGfCfCfCmGfUfUfUmGfUfCmA3′ |
| | (SEQ ID NO: 30)3′- |
| | fCmAfUmAfCmAmAfCmGmGmGfCmAmAmAfCmAmGmU- |
| | Cy5-5′ |
| 110 | (SEQ ID NO: 31)5′-GalNAc-10- |
| | mGfUmAfUmGfUfUmGfCfCfCmGfUfUfUmGfUfCmA3′ |
| | (SEQ ID NO: 32)3′- |
| | fCmAfUmAfCmAmAfCmGmGmGfCmAmAmAfCmAmGmU- |
| | Cy5-5′ |

Note:
N = RNA; dN = DNA; mN = 2′OMe modification; and fN = 2′F modification.

### Example 17. Detection of Cell Targeting Capability of Modified Oligonucleotides

Modified oligonucleotides for animal experiments were filtered through a 0.22 μm membrane before injection.

### 1. Isolation of Mouse Primary Hepatocytes

Mice obtained from Beijing Vital River Laboratory Animal Technology Co., Ltd. were anesthetized. Mouse skin and muscle layer were cut open to expose the liver. Perfusion catheter was inserted into the portal vein, and a small opening was cut in the inferior vena cava to prepare the liver for perfusion. The perfusion Solution I (Hank's, 0.5 mM EGTA, pH 8) and perfusion Solution II (low-glucose DMEM, 100 U/mL Type IV, pH 7.4) were pre-warmed to 40° C. The perfusion solution I (at 37° C.) was infused into the liver along the portal vein at a flow rate of 7 mL/min for 5 minutes until the liver turned gray. Then, the liver was perfused with 37° C. perfusion Solution II at a flow rate of 7 mL/min for 7 minutes. After the perfusion is complete, the liver was isolated and placed in Solution III (10% FBS, low-glucose DMEM, 4° C.) to terminate the digestion. The liver envelope was pierced with forceps, and gently shaken to release the hepatocytes. The hepatocytes were filtered with a 70 μm cell strainer, and then centrifuged at 50 g for 2 minutes. After centrifugation, supernatant was discarded. The hepatocytes were then resuspended in Solution IV (40% percoll low-glucose DMEM, 4° C.), and centrifuged at 100 g for 2 minutes. Supernatant was discarded, and 2% FBS low-glucose DMEM was added to resuspend the cells for subsequence experiments. Trypan blue staining was used to measure cell viability.

### 2. Determination of the GalNAc Binding Curve and Kd Value

The freshly isolated mouse primary hepatocytes were plated into a 96-well plate at 2×10⁴ cells/well (100 μL/well). Different GalNAc-siRNAs were added to each well (see Table 2). The final concentration of each GalNAc-siRNA was 0.9 nM, 2.7 nM, 8.3 nM, 25 nM, 50 nM or 100 nM. The mouse hepatocytes were incubated with GalNAc-siRNAs for 2 hours at 4° C., and then centrifuged at 50 g for 2 minutes. Supernatant was discarded. Next, the hepatocytes were resuspended in 10 μg/ml propidium iodide (PI), stained for 10 minutes, and then centrifuged at 50 g for 2 minutes. The hepatocytes were then washed with cold PBS, followed by centrifugation at 50 g for 2 minutes. Supernatant was discarded and the hepatocytes were resuspended in PBS. The mean fluorescence intensity (MFI) of living cells were measured by a flow cytometer (Beckman). GraphPad Prism 5 software was used to perform nonlinear fitting and calculation of dissociation constant Kd.

The results are listed in Table 3 below, which showed that GalNAc-siRNA can specifically target hepatocytes (or liver cells). The Kd values of the tested GalNAc ligands binding to the cell receptor were determined between 1.5-27.6 nM.

The inhibition constant Ki was also calculated based on the function Ki=IC50/(1+[S]/Km), wherein IC50 stands for inhibitory concentration 50%, [S] is the substrate concentration, and Km is the Michaelis constant. As compared with the preferred galactose ligands disclosed in PCT/US2014/046425 (with Ki values determined between 5.2-51.3 nM, see Example 44), the GalNAc ligands as described herein exhibited higher binding affinities. In addition, GalNAc-siRNAs with different conjugate structures exhibited different receptor binding capabilities. For example, the structures of 101, 104, 108 and 109 exhibited relatively strong receptor binding affinities (the smaller the Kd value, the greater the affinity).

TABLE 3
Kd and Ki values of each experimental group (nM)

| Sample number | 101 | 102 | 103 | 104 | 105 |
|---|---|---|---|---|---|
| Kd | 1.879 | 3.118 | 4.265 | 1.958 | 19.64 |
| Ki | 1.6 | 2.922 | 1.049 | 1.031 | 12.57 |
| Sample number | 106 | 107 | 108 | 109 | 110 |
| Kd | 4.632 | 2.066 | 1.874 | 1.982 | 6.371 |
| Ki | 2.984 | 1.161 | 1.018 | 1.057 | 2.708 |

### Example 18. In Vivo Liver Targeting Test

30 male, 6-7 weeks old specific-pathogen-free Balb/c-nu mice (purchased from Beijing Vital River Laboratory Animal Technology Co., Ltd.) were used. The mice were randomly divided into 6 groups: blank control group, negative control group (or NC1, unconjugated with ligand), test group 1, test group 2, test group 3, and test group 4. The number of mice in each group was 5. The mice were administered by intravenous tail injection, and the dose was about 10 mg/kg (see Table 4 for experimental design). Live imaging of all animals, including white light imaging, was performed before administration, and 15 minutes, 30 minutes, 1 hour, 2 hours, 4 hours, 6 hours after administration. The mice were euthanized 6 hours after administration. The brain, salivary glands, heart, spleen, lungs, liver, kidneys and intestines were isolated for organ imaging (by Xtreme of Bruker Corporation).

TABLE 4
Liver targeting experiment design

| Serial | | Sample | Administration | Administration |
|---|---|---|---|---|
| No. | Group | No. | Dose (mg/kg) | Volume (mL) |
| 1 | Blank | Saline | 0 | 0.2 |
| | control | | | |
| 2 | NC1 | 100 | 10 | 0.2 |
| 3 | Test | 104 | 10 | 0.2 |
| | group 1 | | | |
| 4 | Test | 107 | 10 | 0.2 |
| | group 2 | | | |
| 5 | Test | 108 | 10 | 0.2 |
| | group 3 | | | |
| 6 | Test | 110 | 10 | 0.2 |
| | group 4 | | | |

The in vitro imaging results showed that Sample Nos. 100, 104, 107, 108, and 110 were mainly distributed in the liver, kidney, and gastrointestinal tract, but less in the brain, heart, lungs, spleen and other tissues.

According to the statistical results of the average total photon numbers, Sample Nos. 104, 107, 108 and 110 showed some liver targeting effects, as compared with the 100 group (negative control group). Further, Sample No. 107 and 108 showed statistically significant differences (P<0.001). Sample No. 104 (P<0.01) and Sample No. 110 (P<0.05) also showed statistically significant differences.

TABLE 5
Statistic results of fluorescence intensity values of isolated organs
after subtracting background (average total photon number p/sec)

| | | | | | Left | Right | | Gastrointestinal |
|---|---|---|---|---|---|---|---|---|
| Group | Brain | Heart | Lungs | Liver | kidney | Kidney | Spleen | tract |
| Blank | 1.0E+11 | 3.0E+10 | 1.2E+11 | 3.9E+11 | 5.4E+10 | 6.0E+10 | 7.2E+10 | 4.5E+12 |
| control | | | | | | | | |
| NC1 | 4.1E+11 | 4.3E+11 | 1.6E+12 | 1.4E+13 | 6.3E+12 | 6.6E+12 | 1.4E+12 | 2.5E+13 |
| Test | 4.9E+11 | 4.7E+11 | 1.6E+12 | 3.1E+13 | 7.7E+12 | 8.2E+12 | 1.3E+12 | 2.5E+13 |
| group 1 | | | | | | | | |
| Test | 6.6E+11 | 5.7E+11 | 1.9E+12 | 3.2E+13 | 7.9E+12 | 8.2E+12 | 1.5E+12 | 2.8E+13 |
| group 2 | | | | | | | | |
| Test | 6.3E+11 | 4.6E+11 | 2.1E+12 | 3.3E+13 | 7.0E+12 | 7.2E+12 | 1.1E+12 | 2.5E+13 |
| group 3 | | | | | | | | |
| Test | 5.6 + 11 | 4.1E+11 | 1.8E+12 | 2.9E+13 | 6.9E+12 | 7.8E+12 | 1.1E+12 | 2.3E+13 |
| group 4 | | | | | | | | |

What is claimed is:

1. A compound of Formula (I): 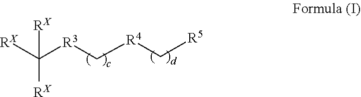
or a pharmaceutically acceptable salt thereof, wherein:
each R^{X} is independently selected from the group consisting of:
H; and
—CH₂OR^{X2}, wherein R^{X2} is H, C₁₋₆ alkyl, or a hydroxyl protecting group; 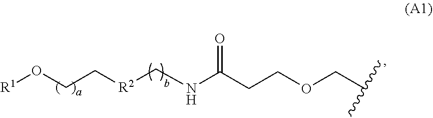
provided that at least one R^{X} is a group of Formula (A1);
each R¹ is an independently selected moiety capable of binding an asialoglycoproteinreceptor (ASGPR);
each R² is independently selected from the group consisting of: —C(R⁶)₂—; —OC(R⁶)₂C(R⁶)₂O—; —C(R⁶)(OH)—C(R⁶)(OH)—; *—C(═O)NR⁷—; *—NR⁷C(═O)—; C₆₋₁₀ arylene; C₂₋₆ alkenylene; and C₂₋₆ alkynylene,
wherein the C₆₋₁₀ arylene, C₂₋₆ alkenylene, and C₂₋₆ alkynylene are each optionally substituted with 1-4 independently selected R^{a}, and the * represents the point of attachment to 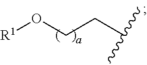
R³ is selected from the group consisting of: 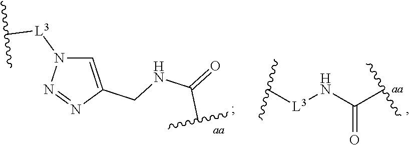
wherein aa represents the point of attachment to 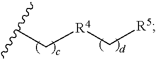
—(CR⁶R⁶)ₓ—O—(CR⁶R⁶)_{y}—, wherein x and y are independently 0, 1, 2, or 3; and
-L³-L^{3C}-, wherein L^{3C} is selected from the group consisting of: C₃₋₁₀ cycloalkylene, C₆₋₁₀ arylene, 5-10 membered heteroarylene, and 4-10 membered heterocyclylene, each of which is optionally substituted with 1-4 independently selected R^{a};
L³ is —C(R⁶)₂—,
R⁴ is selected from the group consisting of: —C(R⁶)₂—; —OC(R⁶)₂C(R⁶)₂O—; —C(R⁶)(OH)—C(R⁶)(OH)—; *—C(═O)NR⁷—; *—NR⁷C(═O)—; C₆₋₁₀ arylene, C₃₋₁₀ cycloalkylene, 5-10 membered heteroarylene, and 4-10 membered heterocyclylene, wherein the C₆₋₁₀ arylene, C₃₋₁₀ cycloalkylene, 5-10 membered heteroarylene, and 4-10 membered heterocyclylene are each optionally substituted with 1-4 independently selected R^{a}, and
wherein the * represents the point of attachment to 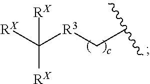
R⁵ is selected from the group consisting of:
hydroxyl; C(O)OH; 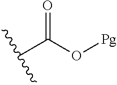
wherein Pg is a carboxyl activating group or a carboxyl protecting group; 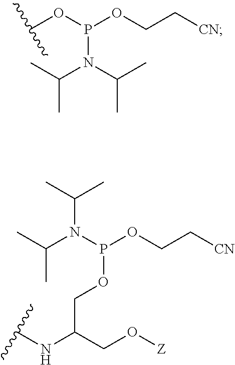
wherein Z is a hydroxyl protecting group; 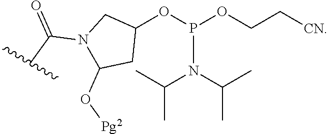
wherein Pg² is a hydroxyl protecting group; and 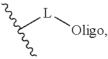
wherein: L is a bond or a divalent group selected from the group consisting of:
—R²—, —R³—, —R⁴—, —O—, —C(═O)—, —C(═O)O—, —OC(═O)—, 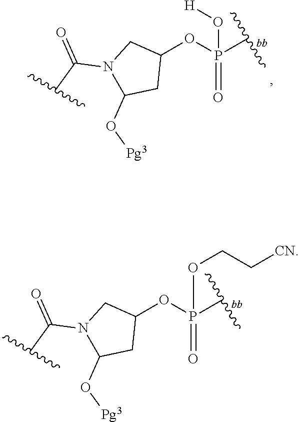
wherein Pg³ is H or Pg²; 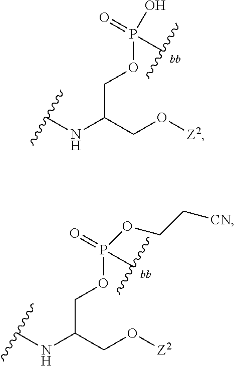
wherein Z² is H or a hydroxyl protecting group; and 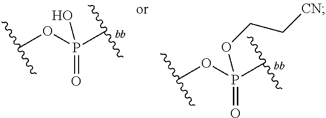
wherein bb represents the point of attachment to Oligo, and
Oligo is an oligonucleotide;
each R⁶ is independently selected from the group consisting of: H; C₁₋₃ alkyl; C₁₋₃ haloalkyl; and halo;
each R⁷ is independently selected from the group consisting of: H; and C₁₋₃ alkyl.
a and b are each independently selected integers from 1 to 10;
c and d are each independently selected integers from 0 to 10; and
each occurrence of R^{a} is independently selected from the group consisting of: halo, cyano, C₁₋₆ alkyl, C₁₋₆ haloalkyl, C₁₋₆ alkoxy, and C₁₋₆ haloalkoxy.

2. The compound of claim 1, wherein at least two R^{X} are each independently a group of Formula (A1);
preferably, each R^{X} is independently a group of Formula (A1).

3. (canceled)

4. The compound of claim 1, wherein
each R¹ is independently a group of Formula (B1) or (B2): 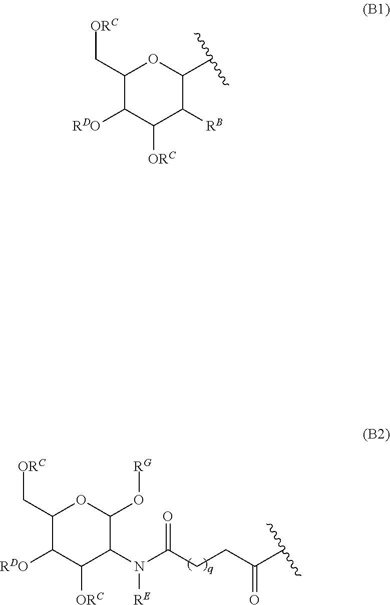
wherein:
R^{D} is selected from the group consisting of R^{C} and 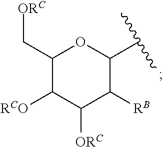
each R^{B} is independently selected from the group consisting of: —NR^{E}R^{F} and —OR^{C};
each R^{C} is independently selected from the group consisting of: H and —C(═O)C₁₋₆ alkyl;
each R^{E} is independently C(═O)C₁₋₆ alkyl;
each R^{F} is independently selected from the group consisting of: H and 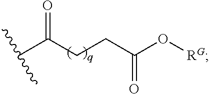
R^{G} is C₁₋₆ alkyl; and
each q is independently an integer selected from 1 to 10;
wherein each R¹ is independently a group having Formula (B1-a), (B1-b), or (B2-a): 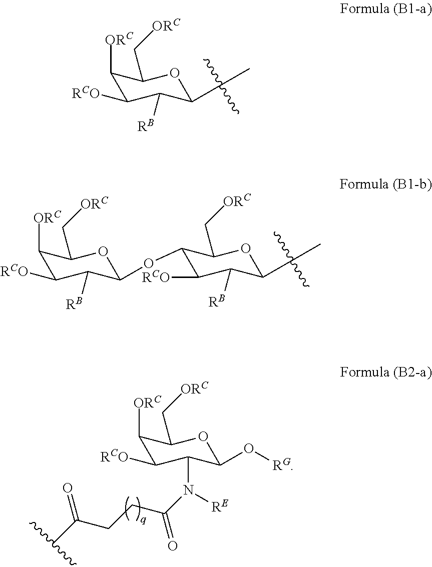
preferably, each R^{B} is independently NR^{E}R^{F};
preferably, each R^{E} is independently —C(═O)C₁₋₆ alkyl;
more preferably, each R^{E} is —C(═O)CH₃;
preferably, each R^{F} is H;
preferably, each R^{F} is 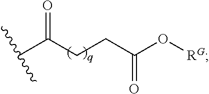
preferably, each R^{B} is NHC(═O)CH₃;
preferably, each R^{B} is 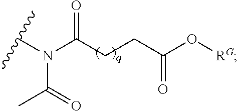
preferably, each R^{B} is independently —OR^{C};
preferably, each R^{C} is independently C(═O)C₁₋₆ alkyl;
more preferably, each R^{C} is C(═O)CH₃;
more preferably, each R^{C} is H;
more preferably, each R^{G} is CH₃.

5-17. (canceled)

18. The compound of claim 1, wherein each R¹ is selected from the group consisting of the following:

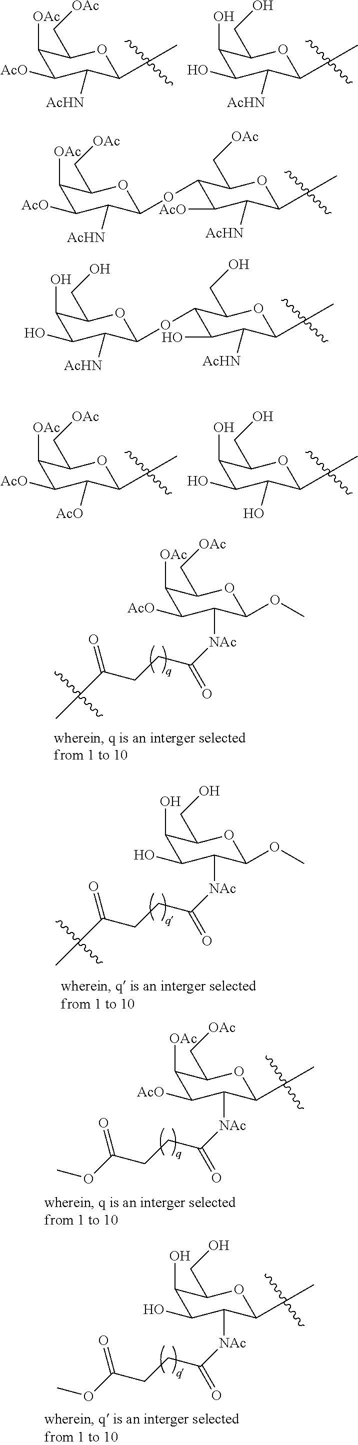

19. The compound of claim 1, wherein each R¹ is the same;
preferably, each R¹ is 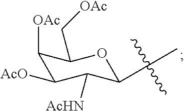 [SMILES: *C1OC(COC(C)=O)C(OC(C)=O)C(OC(C)=O)C1NC(C)=O]
preferably, each R¹ is 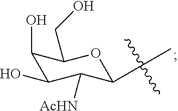 [SMILES: *C1OC(CO)C(O)C(O)C1NC(C)=O]
preferably, each R² is independently *—C(═O)NR⁷— or *—NR⁷C(═O)—;
more preferably, each R² is independently *—C(═O)NR⁷—;
preferably, each R² is *—C(═O)NH—;
preferably, each R² is independently —C(R⁶)₂—;
preferably, each R² is —CH₂—;
more preferably, each R² is the same;
preferably, each a is an independently selected integer from 1 to 4;
more preferably, each a is independently 2 or 3;
preferably, wherein each a is the same;
preferably, each b is an independently selected integer from 1 to 4;
more preferably, each b is independently 2 or 3;
preferably, each b is the same.

20-33. (canceled)

34. The compound of claim 1, wherein each a is the same; each b is the same; and each R² is the same;
preferably, a is an integer from 1 to 4; b is an integer from 1 to 4; and R² is *—C(═O)NR⁷;
more preferably, a is 3; b is 3; and R² is *—C(═O)NH;
preferably, a is an integer from 1 to 4; b is an integer from 1 to 4; and R² is —C(R⁶)₂—;
more preferably, R² is —CH₂—; and 3≤(a+b)≤5.

35-38. (canceled)

39. The compound of claim 1, wherein R³ is 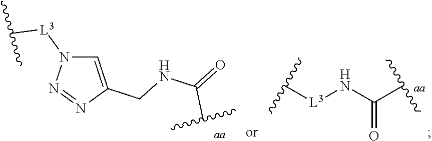
preferably, L³ is —C(R⁶)₂—;
preferably, L³ is —CH₂—;
preferably, c is an integer from 1 to 2;
preferably, c is an integer from 2 to 5;
preferably, c is an integer from 3 to 7;
preferably, R⁴ is *—C(═O)NR⁷—;
preferably, R⁴ is *—C(═O)NH—;
preferably, R⁴ is —C(R⁶)₂—;
preferably, R⁴ is —CH₂—;
preferably, d is an integer from 1 to 2;
preferably, d is an integer from 3 to 7.

40-52. (canceled)

53. The compound of claim 1, wherein R⁴ is —C(R⁶)₂—; and each of c and d is independently 1 or 2;
preferably, R⁴ is —CH₂—; and each of c and d is 1;
preferably, R⁴ is —C(R⁶)₂—; and 4≤(c+d)≤12;
preferably, R⁴ is —CH₂—; and 7≤(c+d)≤10;
preferably, R⁴ is *—C(═O)NR⁷—; and 5≤(c+d)≤10;
preferably, R⁴ is *—C(═O)NR⁷—; and 7≤(c+d)≤9;
preferably, c is 3.

54-59. (canceled)

60. The compound of claim 1, wherein R⁵ is C(O)OH or 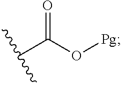
preferably, R⁵ is 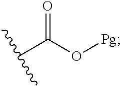
preferably, Pg is a carboxyl activating group;
preferably, wherein Pg is 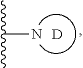
wherein Ring D is a 5-10 membered heteroaryl or 4-10 membered heterocyclyl, each optionally substituted with 1-6 substituents each independently selected from the group consisting of: halo, oxo, NO₂, C(O)C₁₋₄ alkyl, C(O)OC₁₋₄ alkyl, S(O)C₁₋₄ alkyl, C₁₋₆ alkyl, C₁₋₆ haloalkyl, and —OH;
preferably, Pg is: 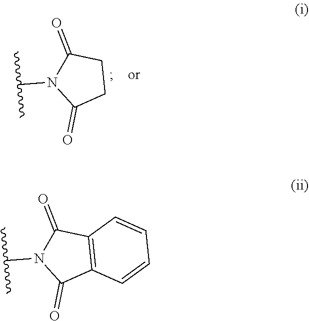 [SMILES: *N1C(=O)CCC1=O ; *N1C(=O)c2ccccc2C1=O]
which is optionally substituted with 1-6 substituents each independently selected from the group consisting of: halo, NO₂, C(O)C₁₋₄ alkyl, C(O)OC₁₋₄ alkyl, S(O)C₁₋₄ alkyl, C₁₋₆ alkyl, C₁₋₆ haloalkyl, and —OH;
preferably, Pg is C₆₋₁₀ aryl or 5-10 membered heteroaryl substituted with 1-6 substituents each independently selected from the group consisting of: —F; —Cl; —NO₂; C(O)C₁₋₄ alkyl, C(O)OC₁₋₄ alkyl, and S(O)C₁₋₄ alkyl;
preferably, Pg is 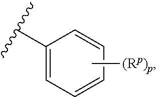
wherein p is an integer from 1 to 5; and
each R^{p} is —F, —Cl, or —NO₂;
preferably, wherein each R^{p} is —F;
preferably, Pg is 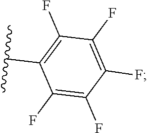 [SMILES: *c1c(F)c(F)c(F)c(F)c1F]
preferably, R⁵ is 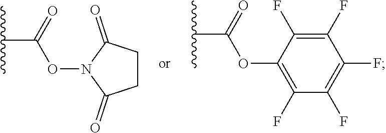 [SMILES: *C(=O)ON1C(=O)CCC1=O ; *C(=O)Oc1c(F)c(F)c(F)c(F)c1F]
preferably, R⁵ is 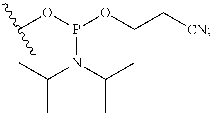 [SMILES: *OP(OCCC#N)N(C(C)C)C(C)C]
preferably, R⁵ is

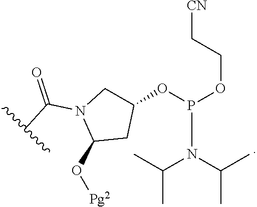

61-71. (canceled)

72. The compound of claim 1, wherein each hydroxyl protecting group is independently selected from the group consisting of: a silyl protecting group; 4-monomethoxytrityl (MMTR); 4,4-dimethoxytrityl (DMTR); and triphenylmethyl (trityl);
preferably, the silyl protecting group is selected from the group consisting of: tert-butyldimethylsilyl (TBMDS); tert-butyldiphenylsilyl (TBDPS), and triisopropylsilyl (TIPS).

73. (canceled)

74. The compound of claim 1, wherein the compound is selected from the group consisting of compounds GalNAc-1 through GalNAc-12.

75. The compound of claim 1, wherein R⁵ is 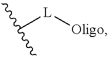
wherein Oligo is an oligonucleotide that is attached to L via the 5′-end, 3′-end, or sequence middle of any strand via a phosphate group;
preferably, L is a bond;
preferably, L is C(═O) or —O—;
preferably, L is 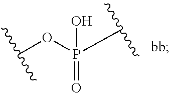 [SMILES: *OP(*)(=O)O]
preferably, L is selected from the group consisting of:

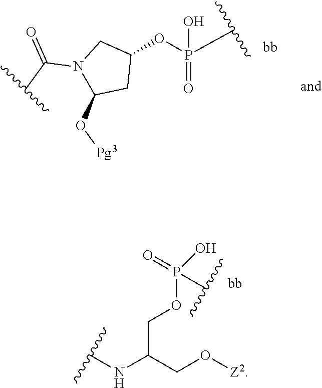

76-79. (canceled)

80. The compound of claim 1, wherein the oligonucleotide comprises a single-stranded oligonucleotide and/or a double-stranded oligonucleotide;
preferably, the oligonucleotide is selected from the group consisting of: DNA, siRNA, miRNA, pre-miRNA, antagomir, mRNA, antisense oligonucleotide (ASO), aptamer, crRNA, tracRNA, and sgRNA.

81. (canceled)

82. The compound of claim 1, wherein the oligonucleotide comprises unmodified nucleotides and/or modified nucleotides;
preferably, the modified nucleotides are each independently selected from the group consisting of: 2′-O-(2-methoxyethyl)-modified nucleotides; 2′-O-alkyl modified nucleotides; 2′-O-allyl modified nucleotides; 2′-C-allyl modified nucleotides; 2′-fluoro modified nucleotides; 2′-deoxy modified nucleotides; 2′-hydroxy modified nucleotides; locked nucleic acids (LNAs) modified nucleotides, glycol nucleic acids (GNAs) modified nucleotides, and unlocked nucleic acids (UNAs) modified nucleotides;
more preferably, wherein the 2′-O-alkyl modified nucleotides are 2′-O-methyl nucleotides.

83-84. (canceled)

85. The compound of claim 1, wherein the oligonucleotide comprises a modifying group, wherein the modifying group is selected from the group consisting of: cholesterol, polyethylene glycol, fluorescent probes, biotin, polypeptides, vitamins, tissue targeting molecules, and a combination thereof;
preferably, the modifying group is a terminal modifying group;
preferably, the phosphate group is a phosphodiester or a modified phosphate group;
preferably, the modified phosphate group is selected from one or more of: thio modified phosphate, amino modified phosphate;
preferably, the thio modified phosphate is phosphorothioate;
preferably, the oligonucleotide comprises one or more peptide nucleic acids and/or morpholino nucleic acids;
preferably, the oligonucleotide is an oligonucleotide of from 5 to 100 base pairs;
preferably, the oligonucleotide conjugate compound is synthesized via solid-phase synthesis or liquid-phase synthesis.

86-92. (canceled)

93. A method for treating and/or preventing pathological conditions or diseases in a subject, wherein the conditions or diseases are caused by the expression of one or more genes in liver cells, the method comprising administering to the subject pharmaceutical composition comprising a compound of claim 75, and a pharmaceutically acceptable excipient.

94. A method for detecting or localizing RNA in the liver of a subject, comprising administering to the subject a a pharmaceutical composition comprising a compound of claim 75, and a pharmaceutically acceptable excipient.

95. The method of claim 93, wherein the one or more genes are selected from: HBV genome, HCV genome, PCSK9, a gene expressing xanthine oxidase (e.g., XDH), URAT1, APOB, liver fibrosis-related genes (e.g., AP3S2, AQP2, AZIN1, DEGS1, STXBP5L, TLR4, TRPM5), genes related to non-alcoholic fatty liver disease (e.g., PNPLA3, FDFT1), and genes related to primary biliary cirrhosis (e.g., HLA-DQB1, IL-12, IL-12RB2);
preferably, the disease or condition is selected from the group consisting of: hereditary angioedema, familial tyrosinemia type I, Alagille syndrome, α-1-antitrypsin deficiency, bile acid synthesis and metabolic defects, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus infection (HBV), hepatitis C virus infection (HCV), steatohepatitis, nonalcoholic steatohepatitis (NASH), nonalcoholic fatty liver disease (NAFLD), hyperglycemia and diseases involving abnormally increased hepatic glucose production similar to type II diabetes, hepatitis, and hepatic porphyrins;
preferably, the compound or pharmaceutical composition is administered intravenously, intramuscularly, subcutaneously, via microneedle patches, orally, via oral or nasal spray, or topically;
preferably, the subject is a mammal;
preferably, the subject is selected from the group consisting of: bovine, equine, sheep, swine, canine, feline, rodent, and primate;
preferably, the subject is human.

96-100. (canceled)

101. A pharmaceutical composition comprising a compound of claim 75 and a pharmaceutically acceptable excipient.

102. The pharmaceutical composition of claim 101, wherein the pharmaceutical composition is formulated in a dosage form selected from the group consisting of: powders, tablets, granules, capsules, solutions, emulsions, suspensions, injections, sprays, aerosols, dry powder inhalations, and microneedle patches;
preferably, the pharmaceutical composition is suitable for administration to a subject in need thereof intravenously, intramuscularly, subcutaneously, via microneedle patches, orally, via oral or nasal spray, or topically;
preferably, the subject is a mammal;
preferably, the mammal is selected from the group consisting of: bovine, equine, sheep, swine, canine, feline, rodent, and primate;
more preferably, the subject is human.

103. (canceled)

104. (canceled)

105. (canceled)

106. (canceled)
